(19) United States
(12) Patent Application Publication
(10) Pub. No.: US 2021/0353660 A1
(43) Pub. Date: Nov. 18, 2021

(54) Pre-Activated Nucleoside IMPDH Inhibitors As Anti-Infective Drugs

(71) Applicant: Octagon Therapeutics Inc., Boston, MA (US)

(72) Inventor: Nathaniel Sherden, Boston, MA (US)

(73) Assignee: Octagon Therapeutics Inc., Boston, MA (US)

(21) Appl. No.: 17/282,526

(22) PCT Filed: Oct. 4, 2019
(86) PCT No.: PCT/US2019/054742

§ 371 Date: Apr. 2, 2021

Related U.S. Application Data
(60) Provisional application No. 62/741,100, filed on Oct. 4, 2018.
(60) Provisional application No. 62/809,953, filed on Feb. 25, 2019.
(60) Provisional application No. 62/811,320, filed on Feb. 27, 2019.

(51) Int. Cl. A61K 31/7056

(52) U.S. Cl. CPC A61K 31/7056

(57) ABSTRACT

The present disclosure provides inosine-5′-monophosphate dehydrogenase (IMPDH)-inhibiting nucleoside derivatives having anti-infective activities, and methods of their synthesis and use.

### REFERENCE TO RELATED APPLICATIONS

This application claims priority to U.S. provisional application 62/741,100, filed Oct. 4, 2018; 62/809,953, filed Feb. 25, 2019; and 62/811,320, filed Feb. 27, 2019, the contents of which are hereby incorporated in its entirety.

### FIELD OF THE INVENTION

The present disclosure relates to medicine and pharmacology. More particularly, the present disclosure is directed to nucleoside analogs for the treatment of infections.

### BACKGROUND

Many major human pathogens have evolved resistance to available antibiotics. More than 700,000 people worldwide die every year due to resistant infections and the healthcare burden in the US, alone, exceeds $20 billion. As the rate of novel antibiotic discovery has slowed, the threat of incurable infections by multidrug-resistant pathogens has risen rapidly. Since 2008, there has been up to a 600% increase in certain resistant infections. For example, 20% of Pseudomonas aeruginosa infections and 54% of Acinetobacter baumannii infections are multidrug-resistant. There is a critical need for new antibiotics, particularly against the ESKAPE group pathogens. At the same time the discovery of novel antibiotics has become increasingly challenging, prompting many drug companies to abandon their antibiotics development programs.

Historically, antibiotic discovery has relied on screening of libraries of natural products and synthetic compounds for inhibition of bacterial growth in vitro. Standard growth media in these screens contain diverse mixtures of nutrients including all or most amino acids, sugars, and nucleic acids. While this approach has yielded essentially all current antibiotics, its success rate has declined precipitously in recent years. Other recent efforts have focused on the identification of inhibitors of essential bacterial gene products, but some have failed, prompting major drug companies to abandon their antibiotic development programs.

Accordingly, what is needed are new anti-infective compounds and formulations to inhibit the growth of problematic infective organisms, including multidrug-resistant infections.

Also needed are new formulations to treat fungal, yeast, and parasite infections.

### SUMMARY

It has been determined that that certain IMPDH-inhibiting nucleosides with substitutions at their ribose 5′-position are effective anti-infective drugs. These differentially 5′-derivatized analogs require no activation, are not inactivated or catabolized by phosphatases, and are more conducive to being taken up by and accumulated in, certain infective organisms relative to their corresponding nucleotide or nucleoside.

These discoveries have been exploited to develop the present invention, which, in one aspect, provides an anti-infective formulation comprising a nucleoside analog inhibitor of inosine monophosphate dehydrogenase (IMPDH) and a pharmaceutically acceptable carrier.

In some embodiments, the nucleoside analog inhibitor is a compound of Formula 1

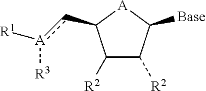

or a pharmaceutically acceptable salt thereof,
wherein:
Base is selected from the group consisting of

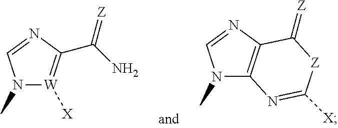

A is selected from the group consisting of —CH—, —CH₂—, —N—, —NH—, —O—, —SO₂R³—, and —S—;
W is selected from the group consisting of —C—, —CH—, —CH₂—, —N—, and —NH₂—;
X is, independently at each occurrence, selected from the group consisting —OH, —SH, —NH₂ halogen;
Y is selected from the group consisting of —OH, —SH, —NH₂, and —N₃;
Z is selected from the group consisting of O, S, and;
R¹ is selected from the group consisting of —PA₁O₂(R³)₂, — and —SO₂R³;
R² is, independently at each occurrence, selected from the group consisting of —OH, —NH₂, and —N₃;
R³ is, independently at each occurrence, selected from the group consisting of —H, —C₁₋₆ alkyl, —C₁₋₆ alkenyl, —C₁₋₆ allyl —C₆₋₁₀ aryl, and —N(R⁴)₂, wherein —C₁₋₆ alkyl is optionally substituted with one or more halo;
R⁴ is, independently at each occurrence, selected from the group consisting of —H, —C₁₋₆ alkyl, —C₁₋₆ alkenyl, —C₁₋₆ allyl and —C₆₋₁₀ aryl; and

[symbol] is an optional bond, and

wherein the derivative is not:

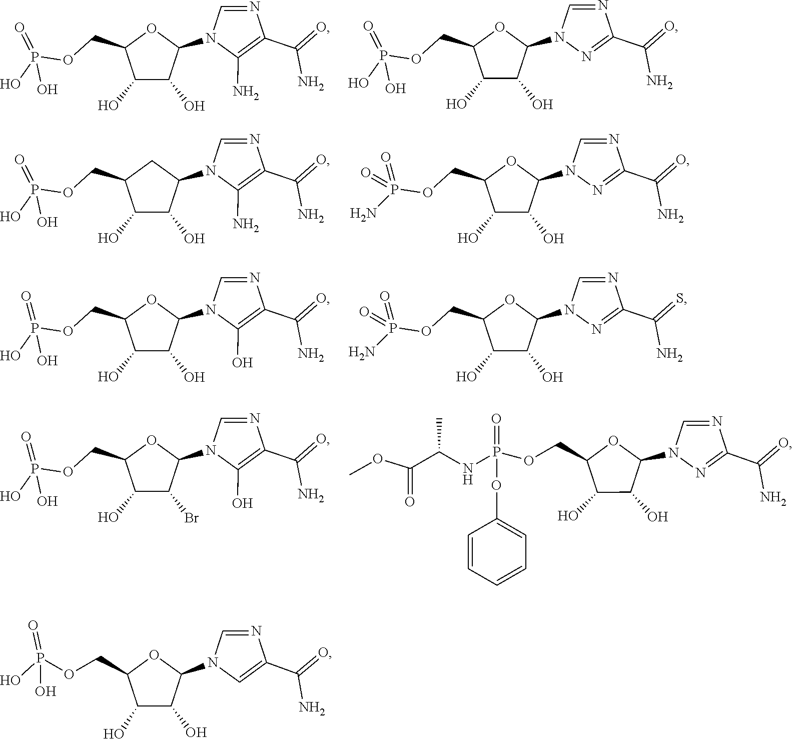

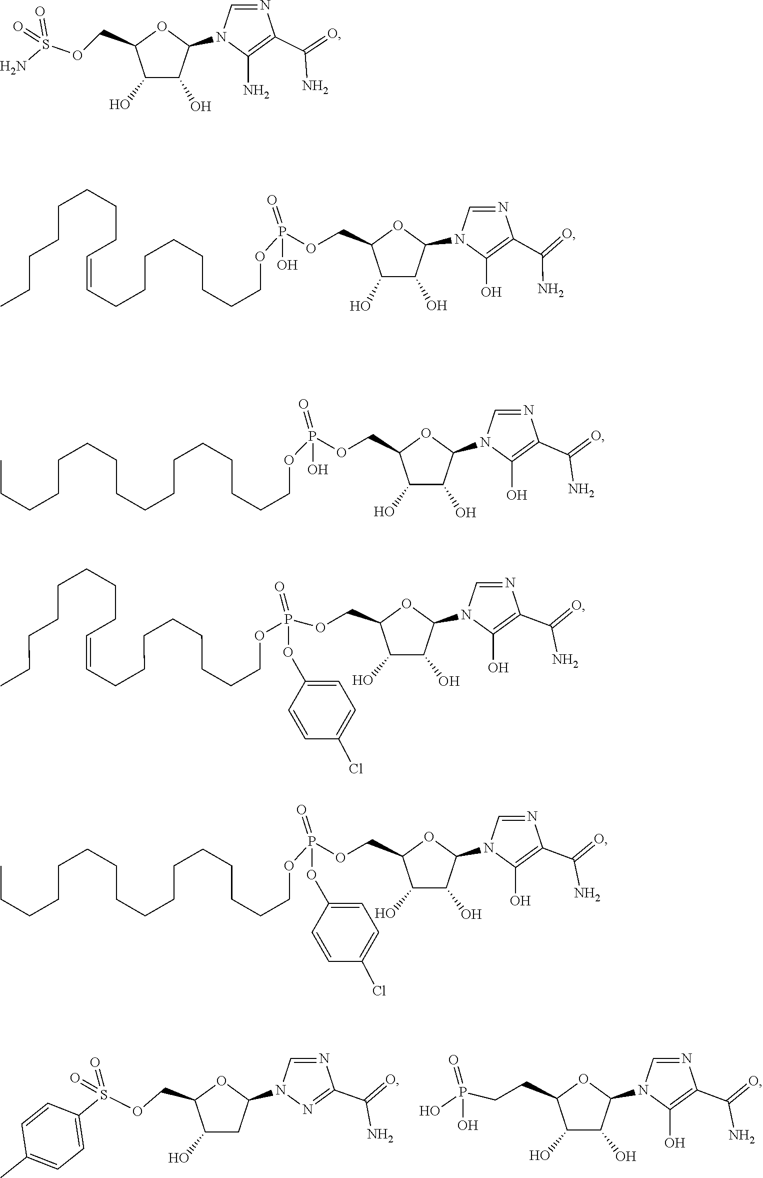

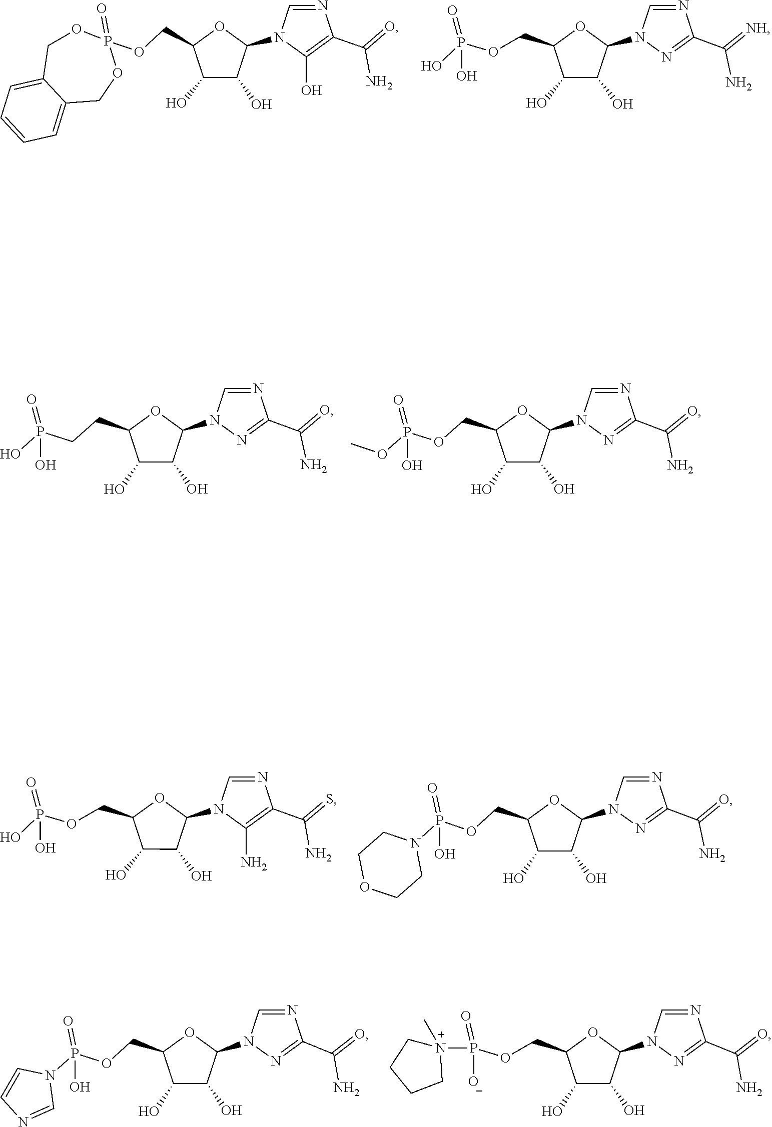

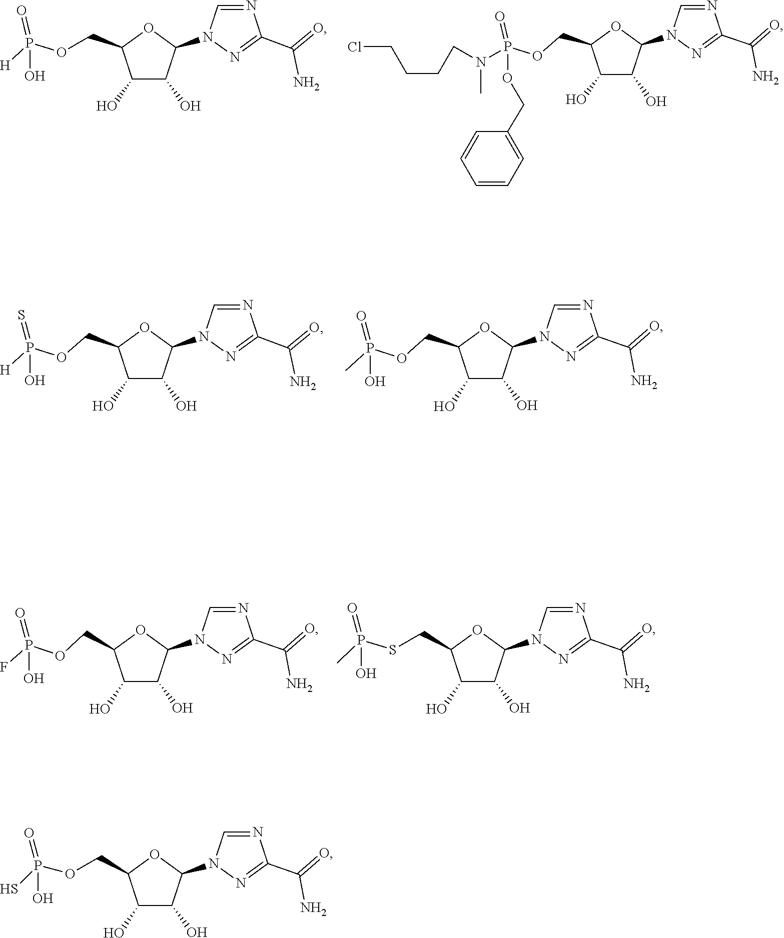

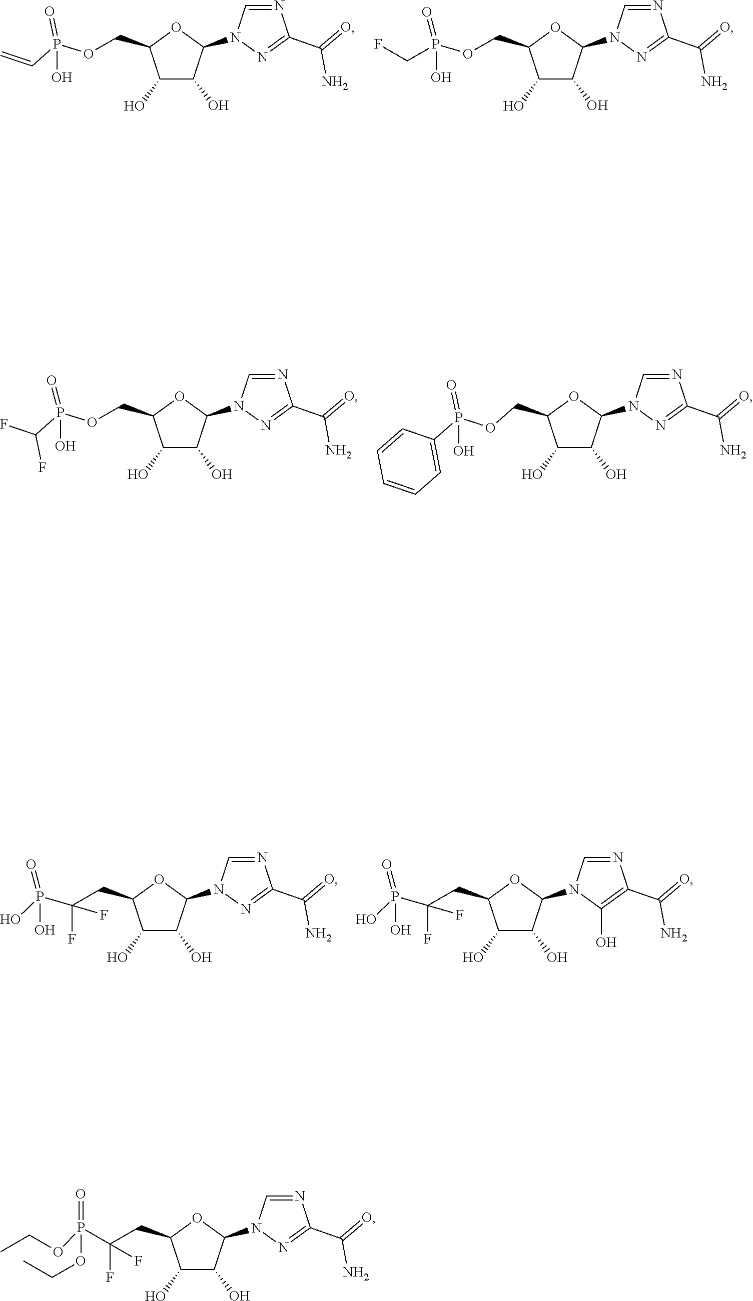

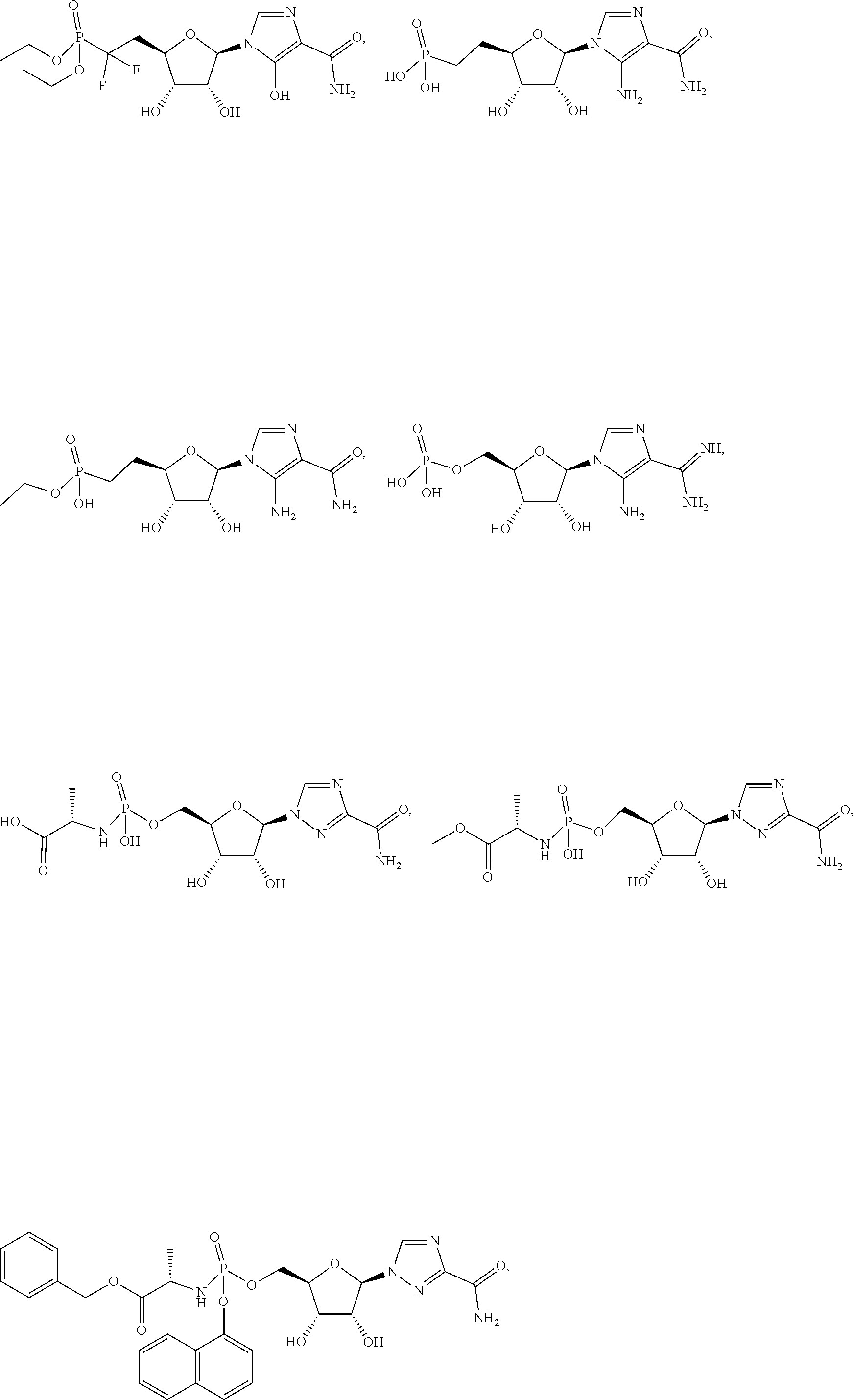

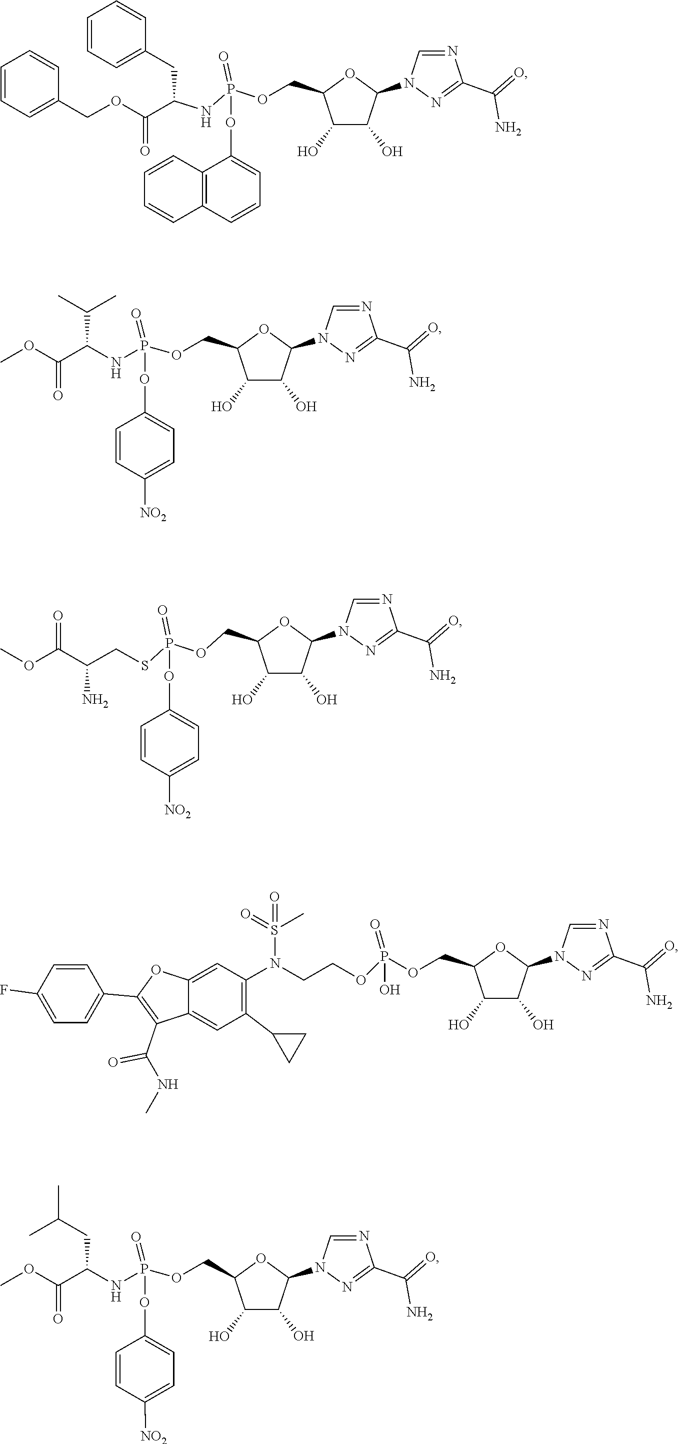

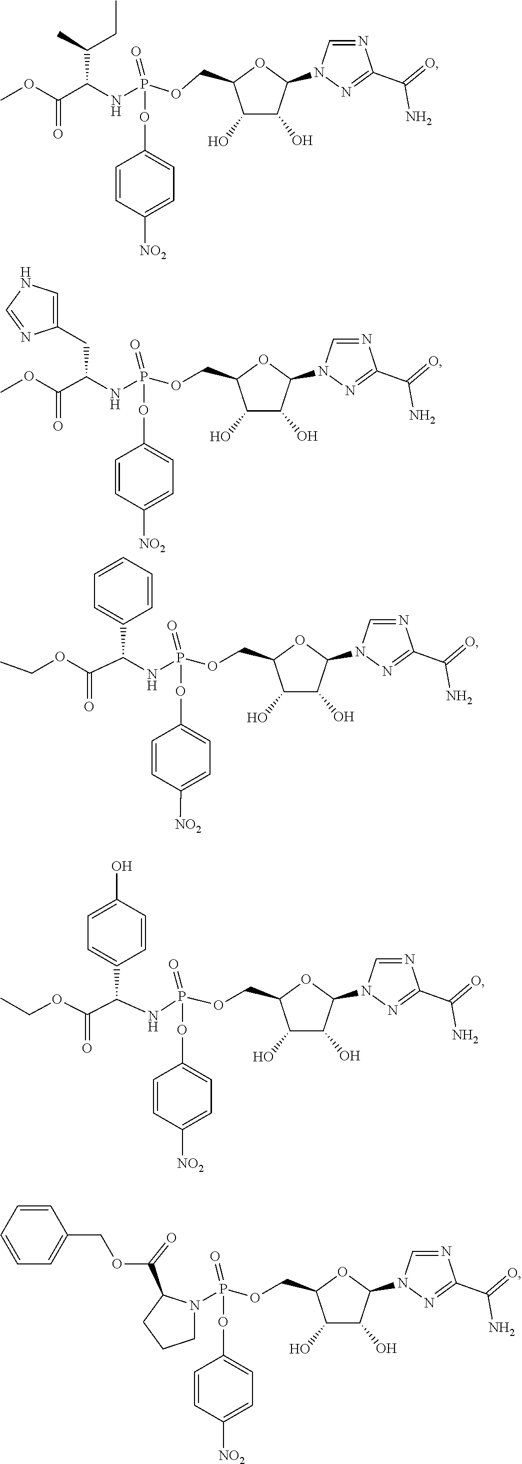

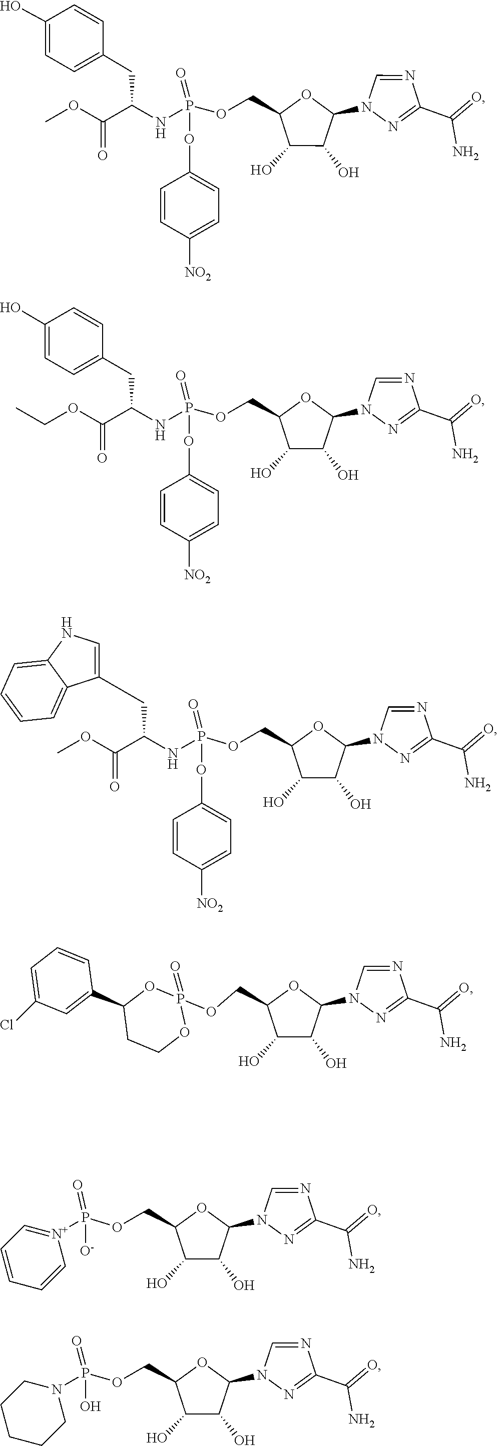

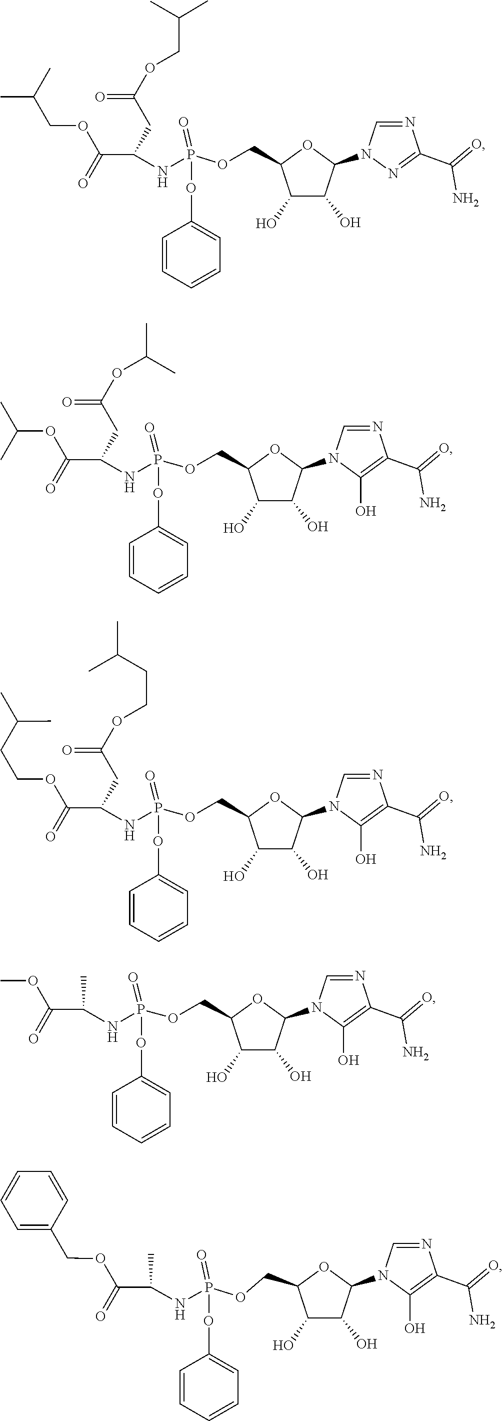

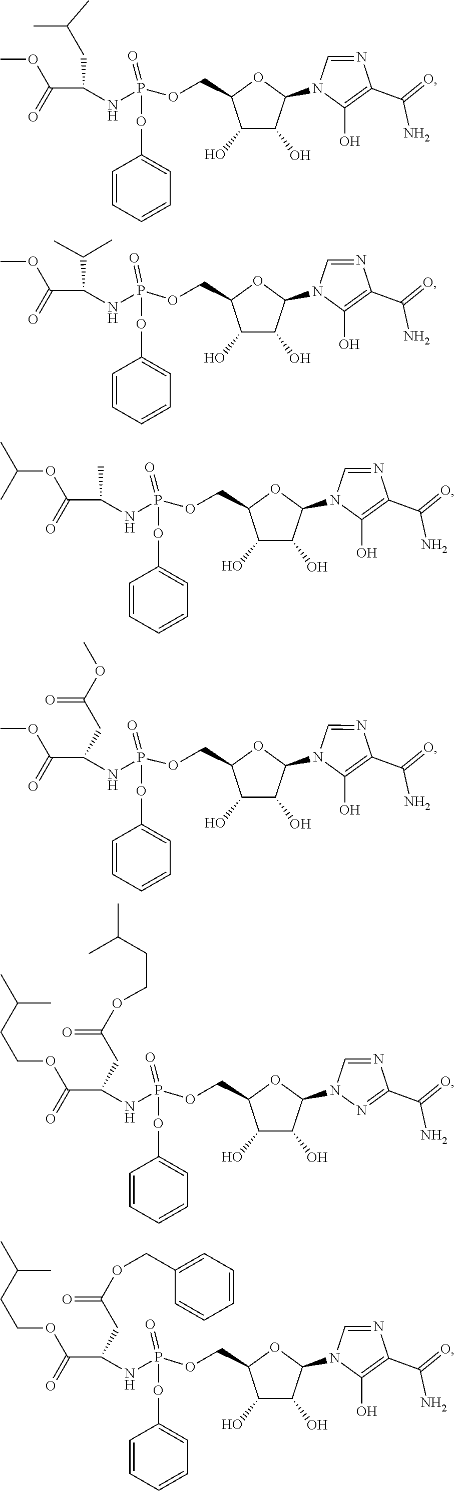

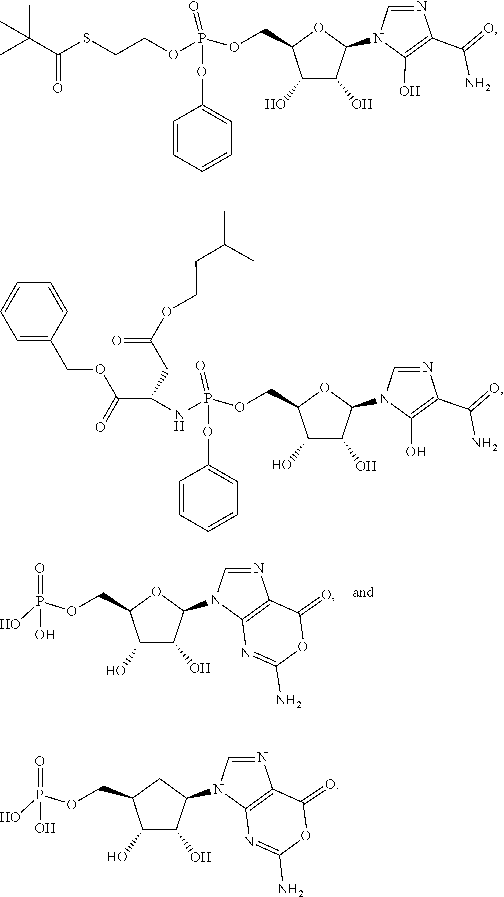

In particular embodiments, the anti-infective formulation comprises a nucleoside analog inhibitor having Formula II:

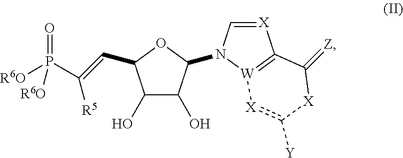

a compound of Formula III:

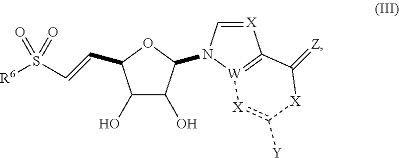

a compound of Formula IV:

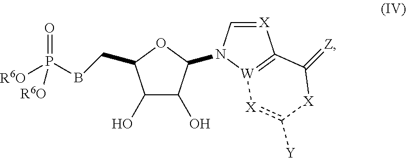

a compound of Formula V:

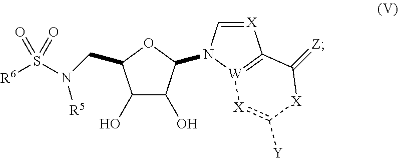

or a pharmaceutically acceptable salt of a compound of Formula II, Formula III, Formula IV, or Formula V, wherein:

W is selected from the group consisting of —C—, —CH₂—, —N—, and —NH₂—;

X is, independently at each occurrence, selected from the group consisting of —O—, —OH, —S—, —SH, —NH—, —NH₂, —CH₂—, and —CH₃;

Y is selected from the group consisting of —OH, —SH, —NH2, and —N3;

Z is selected from the group consisting of O, S, and NH;

B is selected from the group consisting of S, O, NH, and NR⁵.

R⁵ is selected from the group consisting of —H, halo, —C₁₋₆ alkyl, —C₁₋₆ alkenyl, and —C₆₋₁₀ aryl;

R⁶ is, independently at each occurrence, selected from the group consisting of —H, —C₁₋₆ alkyl, —C₁₋₆ alkenyl, —C₆₋₁₀ aryl, —CH₂—C₆₋₁₀ aryl, —O—C₆₋₁₀ aryl, —N(C₁₋₆ alkyl)₂, —NH(C₁₋₆ alkyl), and —NH₂, wherein —C₆₋₁₀ aryl is optionally substituted with one or more R⁷ or alternatively, each R⁶, together with the atom to which they are attached, can form C₃₋₁₂ heterocycle or C₃₋₁₂ heteroaryl, wherein heterocycle or heteroaryl is optionally substituted with one or more R⁷;

R⁷ is selected from the group consisting of halo, —C₁₋₆ alkyl, —C₁₋₆ alkenyl, —C₆₋₁₀ aryl, —OC₁₋₆ alkyl, —N(C₁₋₆ alkyl)₂, —NH(C₁₋₆ alkyl), —NH₂, and —OH; and

[symbol]

is an optional bond.

In specific embodiments, the compound of Formula I, II, III, IV, or V is a compound having a structure selected from the group consisting of:

| Structure | |
|---|---|
| No. | Structure |
| 1 | 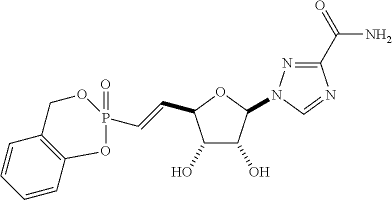 |
| 2 | 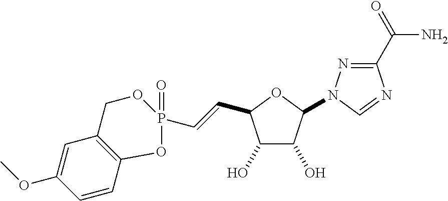 |
| 3 | 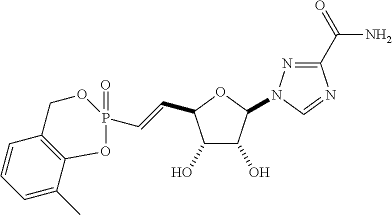 |
| 4 | 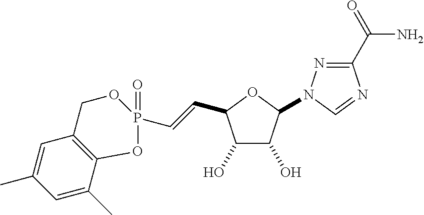 |
| 5 | 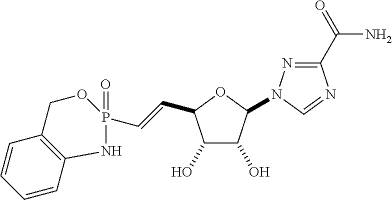 |
| 6 | 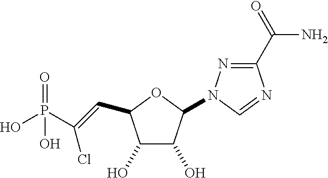 |
| 7 | 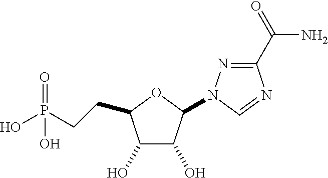 |
| 8 | 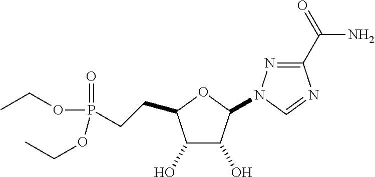 |
| 9 | 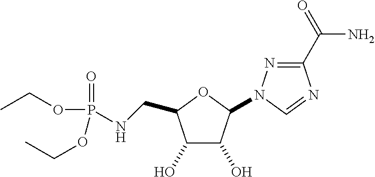 |
| 10 | 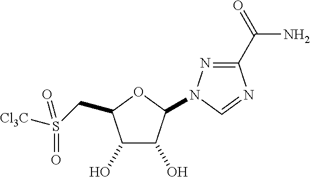 |
| 11 | 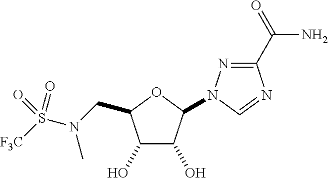 |
| 12 | 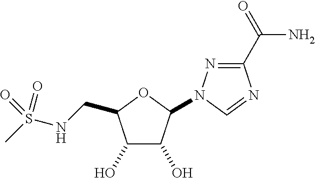 |
| 13 | 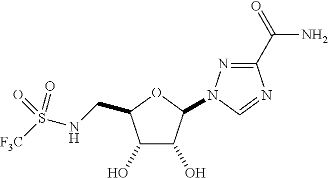 |
| 14 | 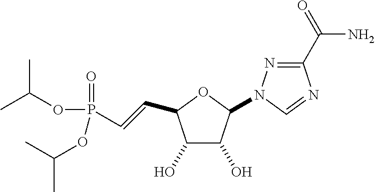 |
| 15 | 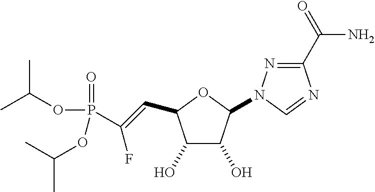 |
| 16 | 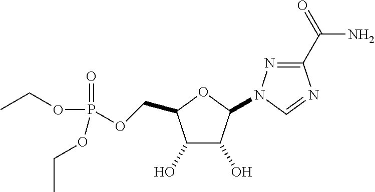 |
| 17 | 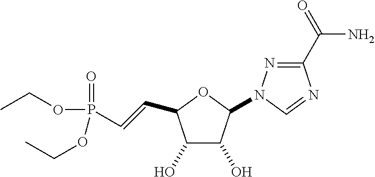 |
| 18 | 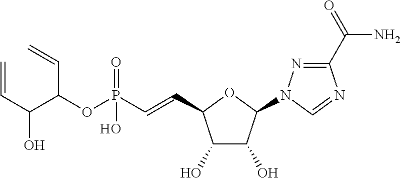 [SMILES: C=CC(O)C(C=C)OP(=O)(O)/C=C/[C@H]1O[C@@H](n2cnc(C(N)=O)n2)[C@H](O)[C@@H]1O] |
| 19 | 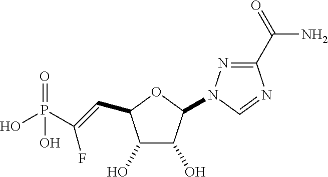 |
| 20 | 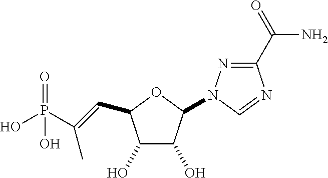 |
| 21 | 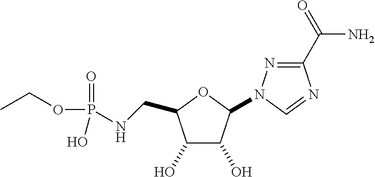 |
| 22 | 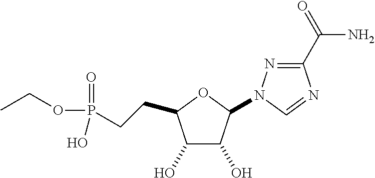 |
| 23 | 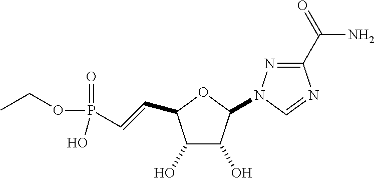 |
| 24 | 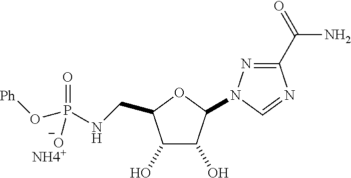 |
| 25 | 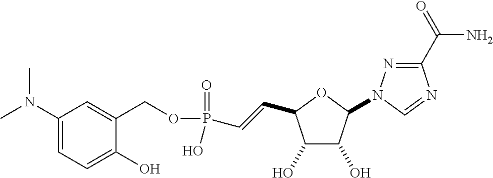 |
| 26 | 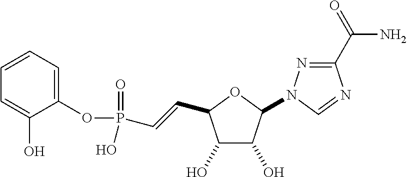 |
| 27 | 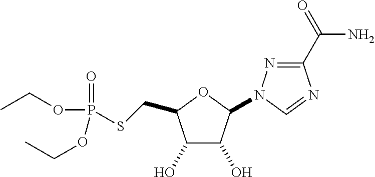 |
| 28 | 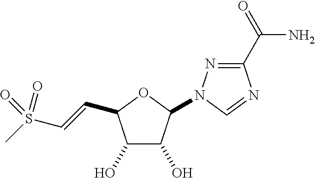 |
| 29 | 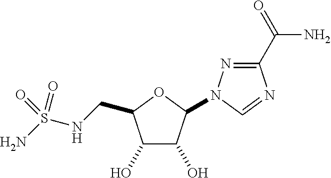 |
| 30 | 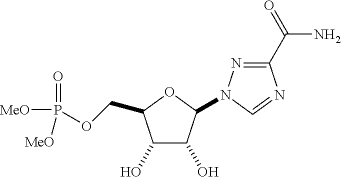 |
| 31 | 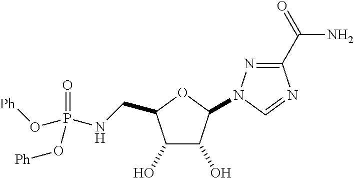 |
| 32 | 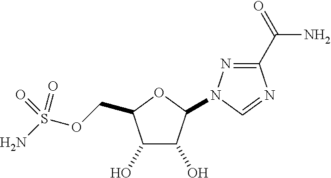 |
| 33 | 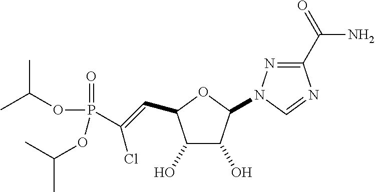 |
| 34 | 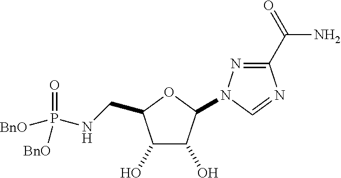 |
| 35 | 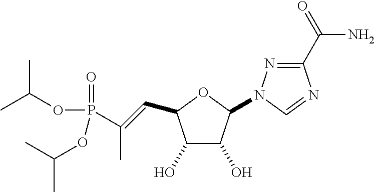 |
| 36 | 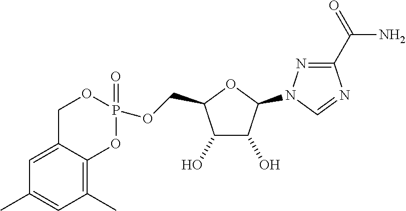 [SMILES: Cc1cc(C)c2c(c1)COP(=O)(OC[C@H]1O[C@@H](n3cnc(C(N)=O)n3)[C@H](O)[C@@H]1O)O2] |

or a pharmaceutically acceptable salt thereof.

In another aspect, the disclosure provides a method of treating an infection in a mammal, comprising administering to the mammal a therapeutically effective amount of the anti-infective formulation such that the infection is reduced, the formulation comprising a nucleoside analog inhibitor of inosine monophosphate dehydrogenase (IMPDH) and a pharmaceutically acceptable carrier, the inhibitor not being a known anti-infective compound.

In some embodiments, the nucleoside analog inhibitor has the structure of Formula I, Formula II, Formula III, Formula IV, or Formula V, as described above. In particular embodiments, the nucleoside analog inhibitor has a structure selected from structures 1-36 above.

In certain embodiments, the infection being treated is a bacterial infection, a fungal infection, a viral infection, a yeast infection, a multicellular parasitic infection, or a protozoan infection.

In particular embodiments, the infection is a bacterial infection such as a Gram positive or Gram negative bacterial bacteria. In certain embodiments, the bacterial infection is an infection by Mycobacterium tuberculosis, Mycobacterium avium, Mycobacterium intracellulare, or Mycobacterium leprae.

In yet another aspect, the disclosure provides a method of inhibiting the growth and/or proliferation of an infective organism, comprising contacting the organism with a growth and/or proliferation-inhibiting amount of a formulation comprising a nucleoside analog derivative of Formula I, II, III, IV, or V, wherein the derivative is not one of the following anti-infective compounds:

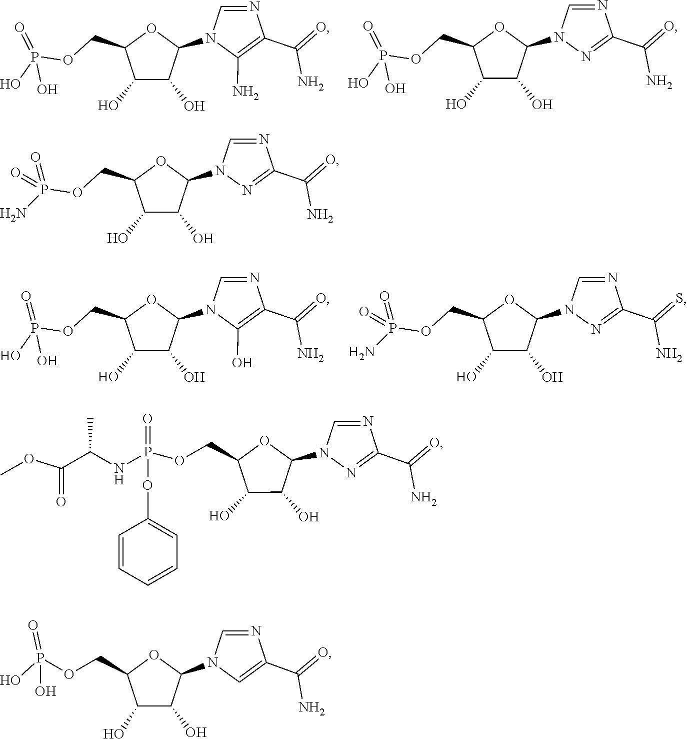

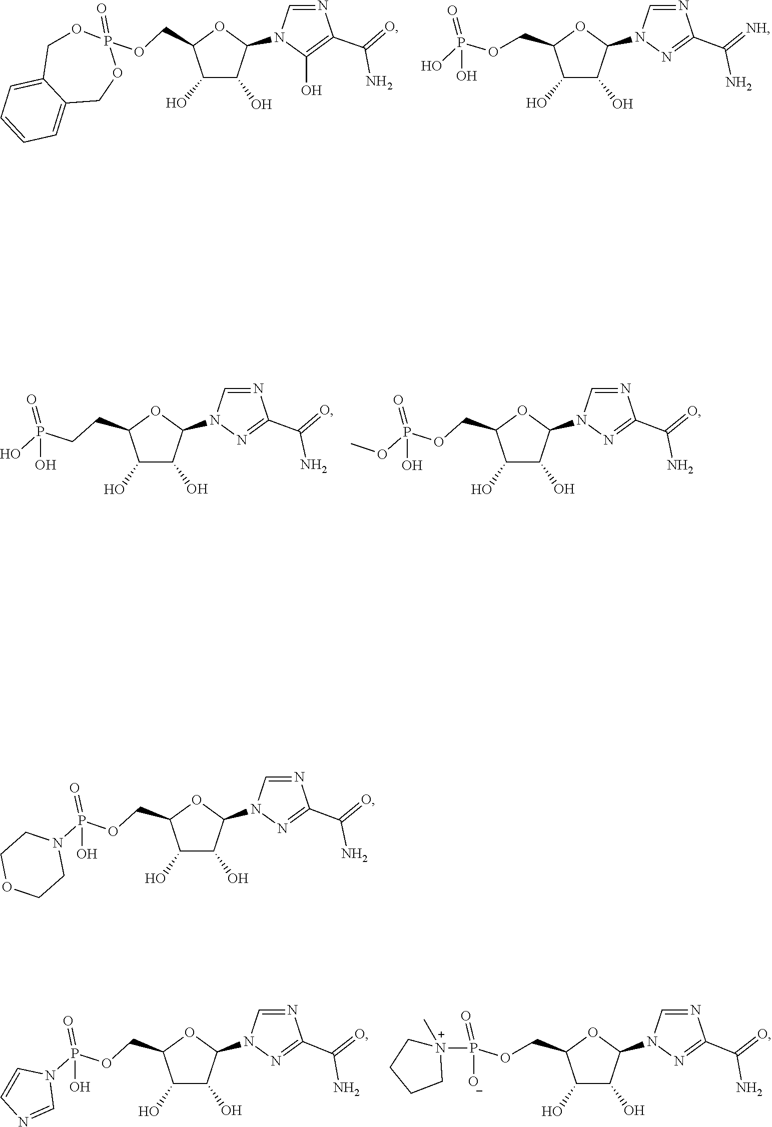

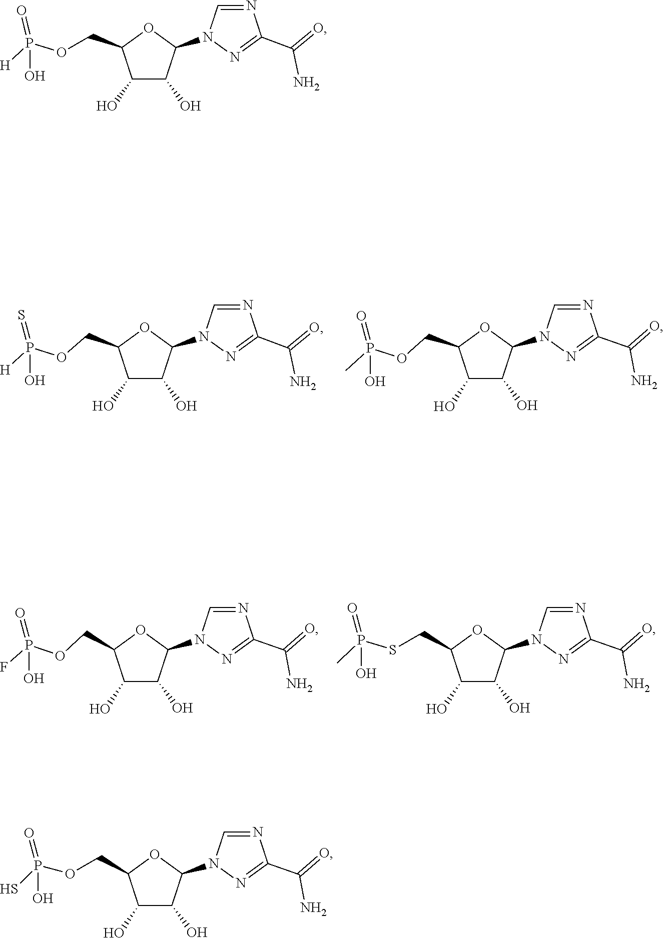

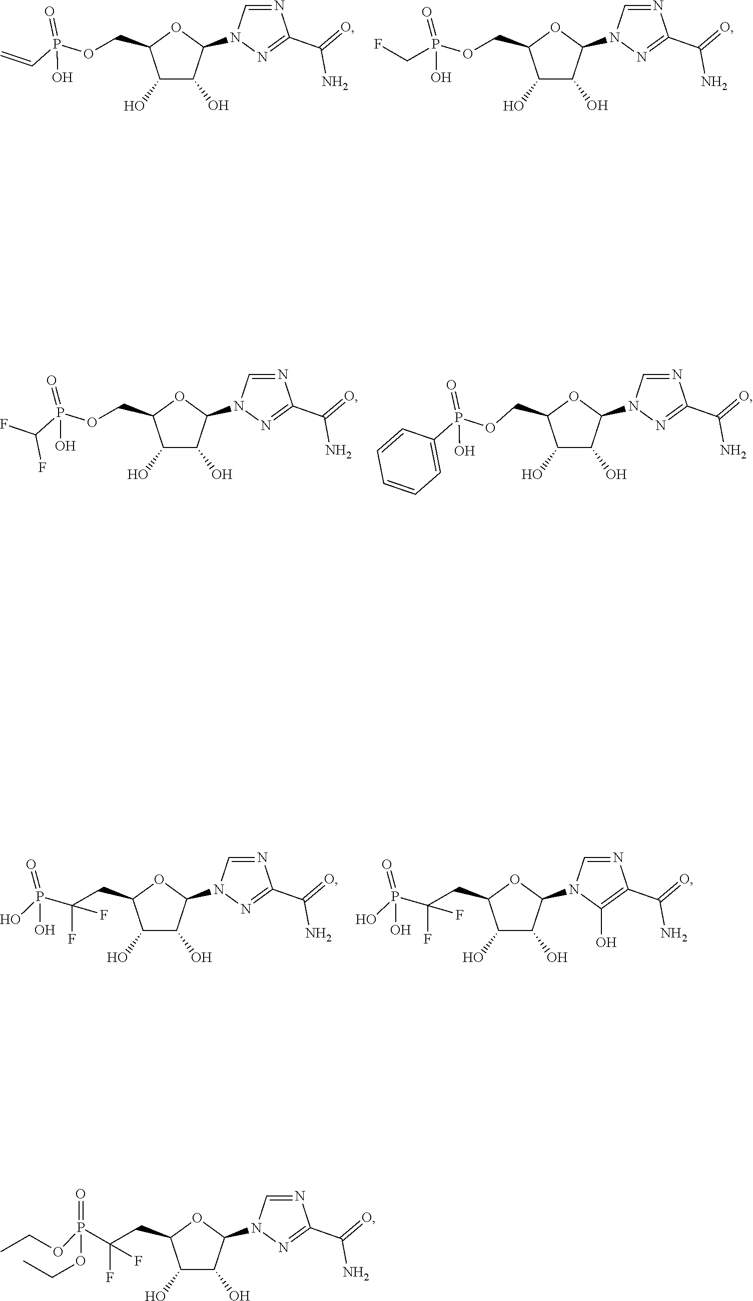

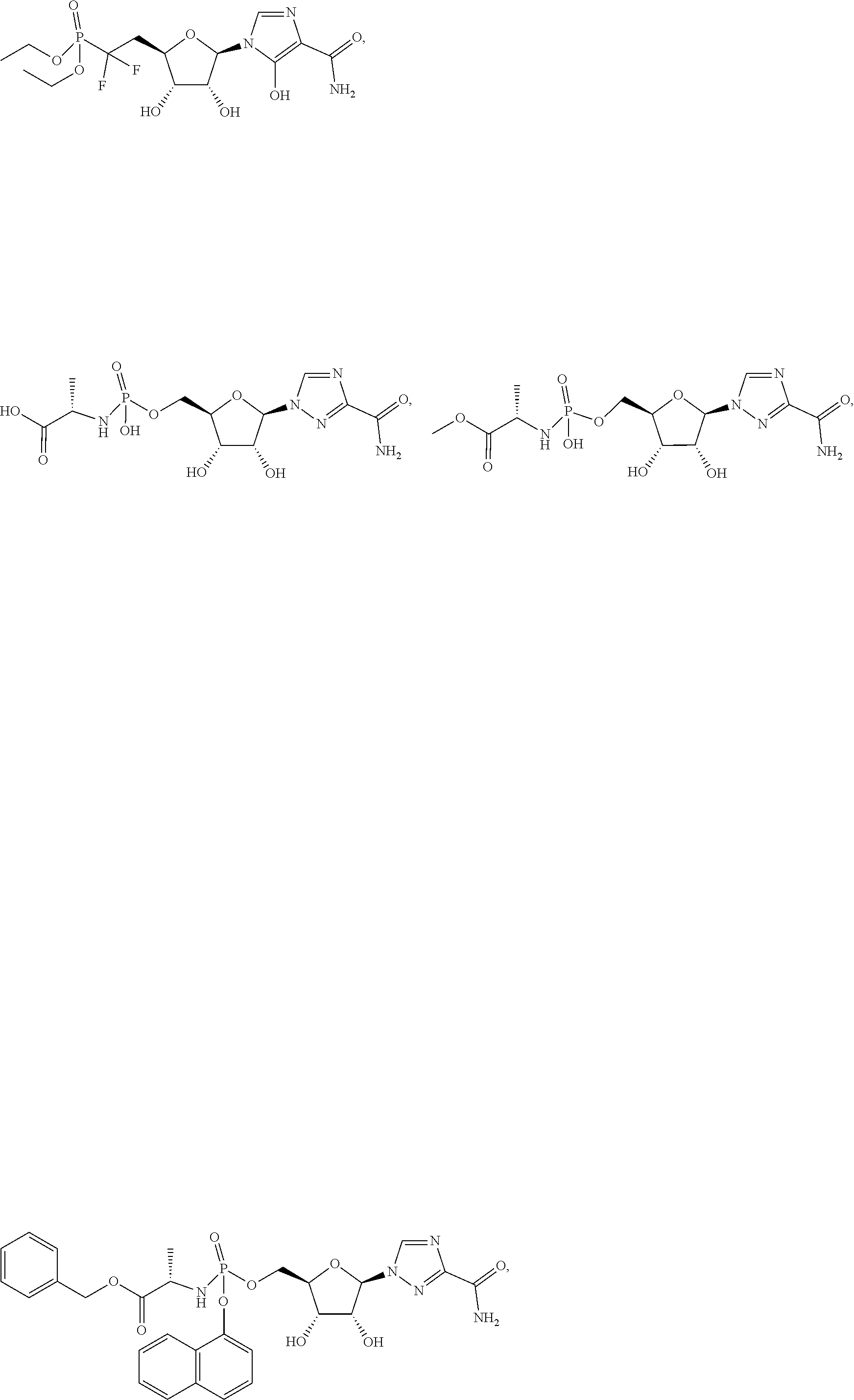

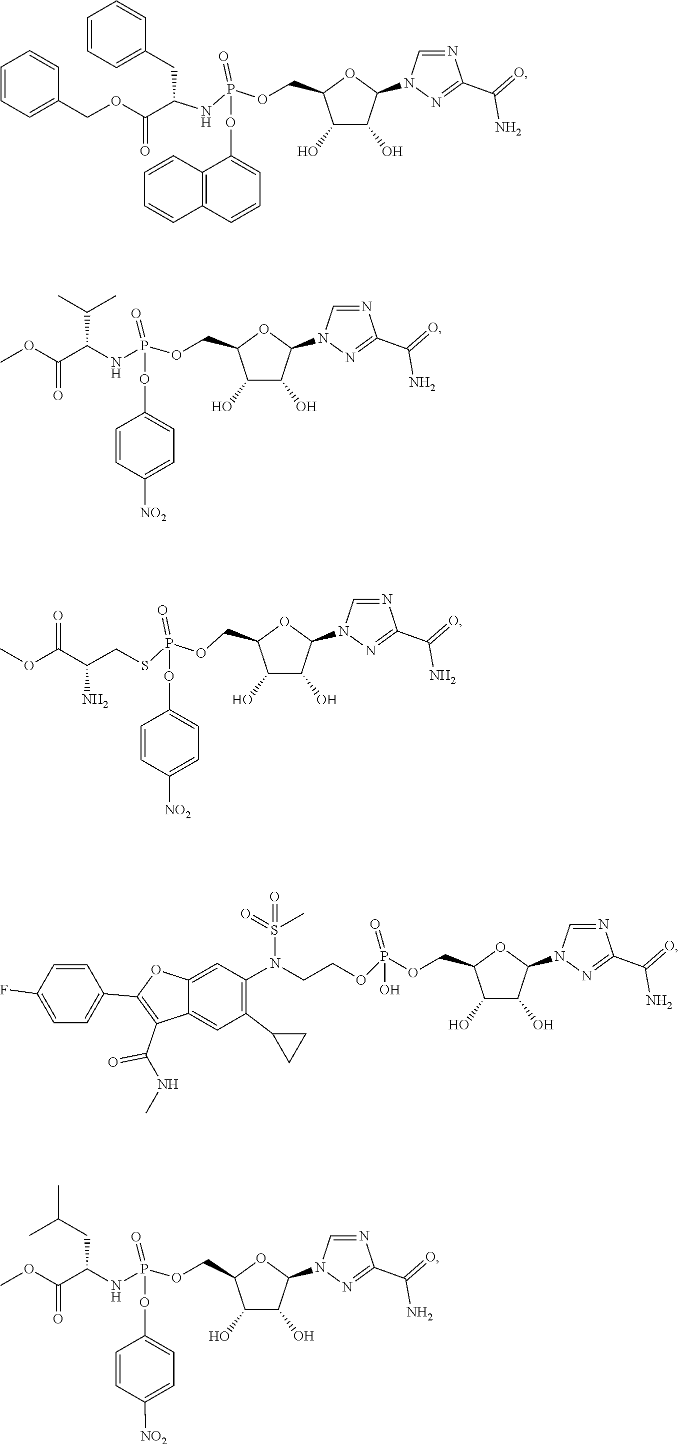

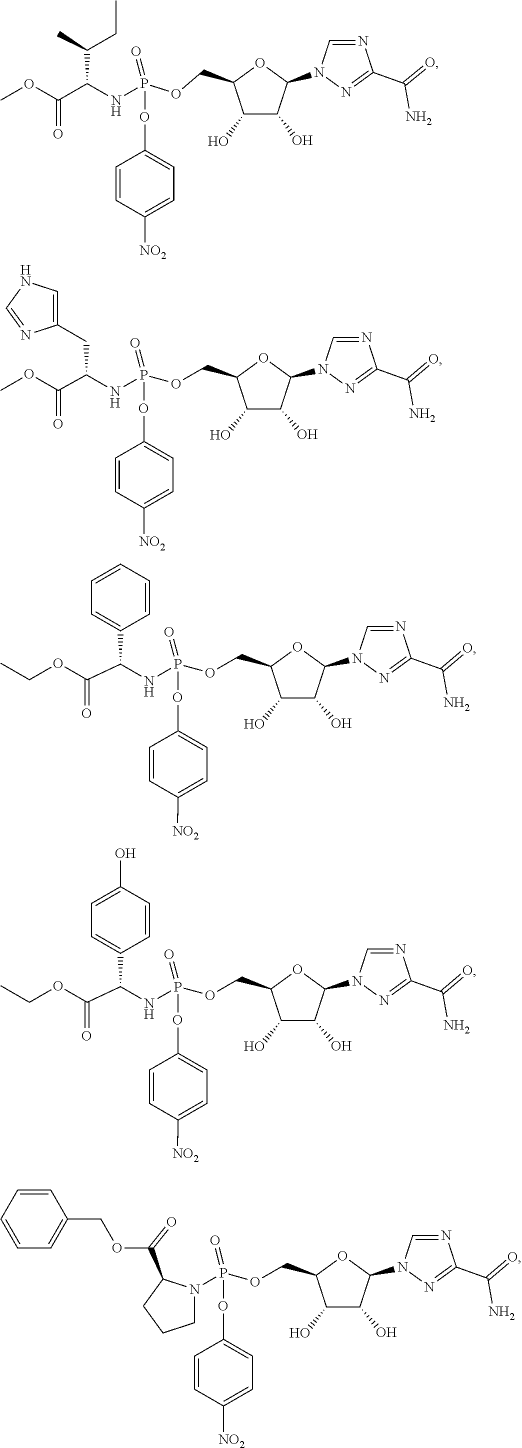

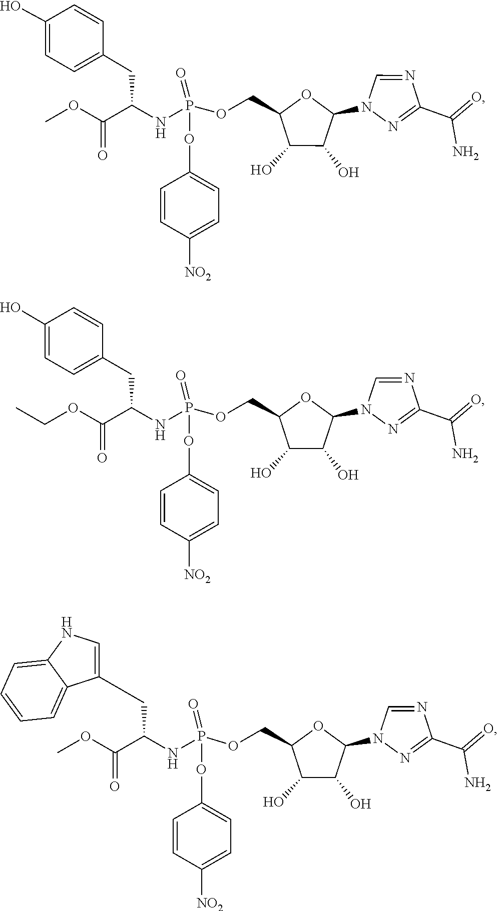 [SMILES: COC(=O)[C@H](Cc1ccc(O)cc1)NP(=O)(OC[C@H]1O[C@@H](n2cnc(C(N)=O)n2)[C@H](O)[C@@H]1O)Oc1ccc([N+](=O)[O-])cc1 ; CCOC(=O)[C@H](Cc1ccc(O)cc1)NP(=O)(OC[C@H]1O[C@@H](n2cnc(C(N)=O)n2)[C@H](O)[C@@H]1O)Oc1ccc([N+](=O)[O-])cc1 ; COC(=O)[C@H](Cc1c[nH]c2ccccc12)NP(=O)(OC[C@H]1O[C@@H](n2cnc(C(N)=O)n2)[C@H](O)[C@@H]1O)Oc1ccc([N+](=O)[O-])cc1]

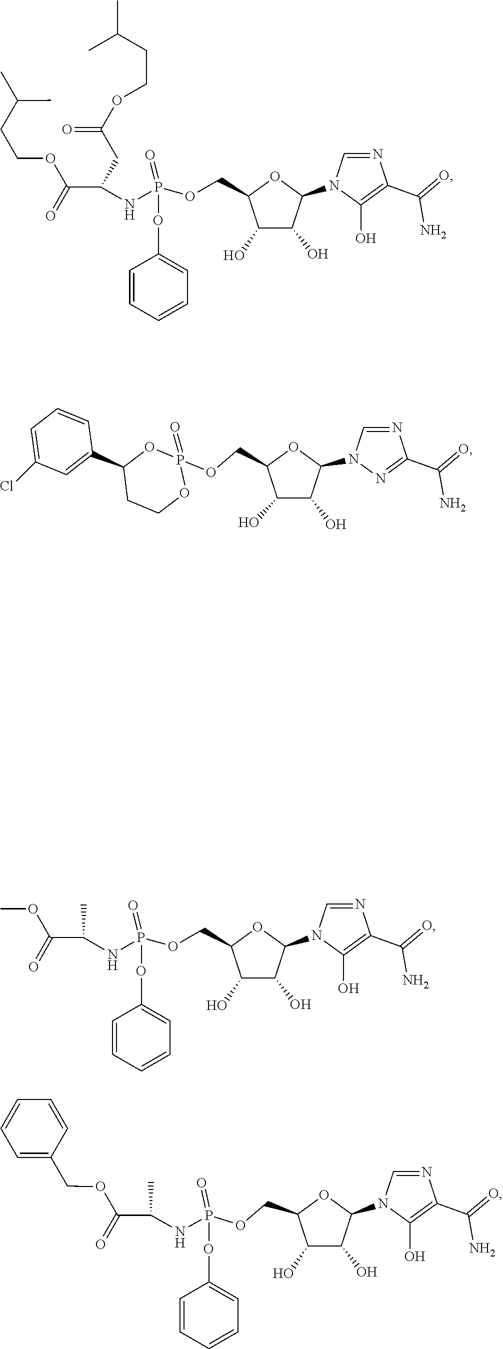 [SMILES: CC(C)CCOC(=O)C[C@H](NP(=O)(OC[C@H]1O[C@@H](n2cnc(C(N)=O)c2O)[C@H](O)[C@@H]1O)Oc1ccccc1)C(=O)OCCC(C)C ; NC(=O)c1ncn([C@@H]2O[C@H](COP3(=O)OCC[C@@H](c4cccc(Cl)c4)O3)[C@@H](O)[C@H]2O)n1 ; COC(=O)[C@H](C)NP(=O)(OC[C@H]1O[C@@H](n2cnc(C(N)=O)c2O)[C@H](O)[C@@H]1O)Oc1ccccc1 ; C[C@H](NP(=O)(OC[C@H]1O[C@@H](n2cnc(C(N)=O)c2O)[C@H](O)[C@@H]1O)Oc1ccccc1)C(=O)OCc1ccccc1]

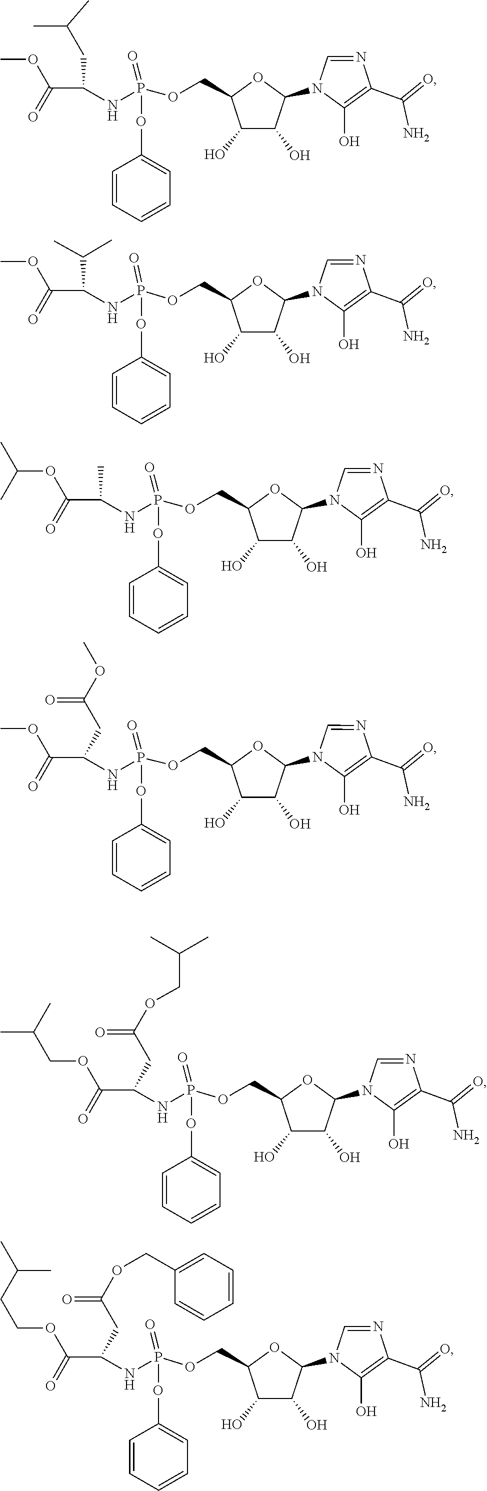

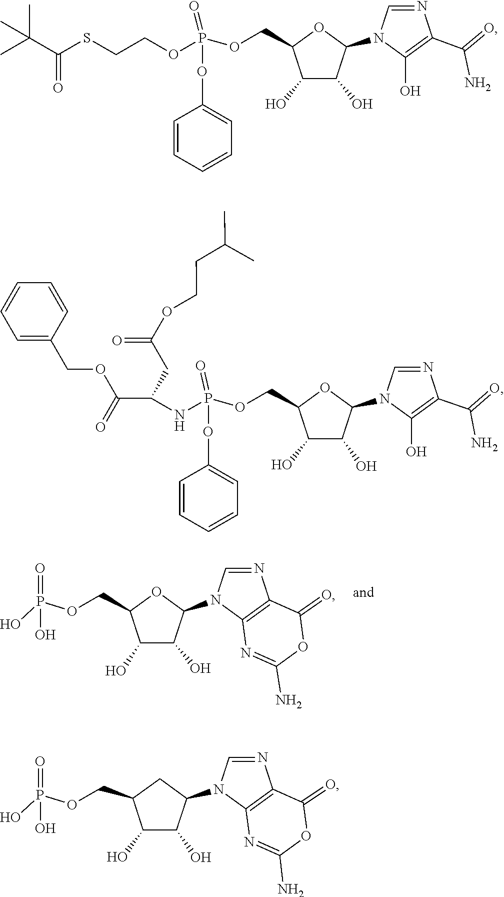

and wherein the infective organism is a bacterium, a fungus, a yeast, a multicellular parasite, or a protozoan.

### DESCRIPTION

The disclosures of patents, patent applications, and publications in their entireties are hereby incorporated by reference into this application to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 20% or 10%, including ±5%, +1%, and +0.10% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with an infection an effective amount of a anti-infective formulation of the disclosure for conditions related to infections.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition”, “pharmaceutical composition”, or “formulation” refers to a nucleoside analog or derivative inhibitor according to the disclosure in a pharmaceutically acceptable carrier.

An “oral dosage form” includes a unit dosage form prescribed or intended for oral administration.

As used herein, the term “alkyl,” by itself or as part of another substituent means, unless otherwise stated, a straight or branched chain hydrocarbon having the number of carbon atoms designated (i.e., C1-C6-alkyl means an alkyl having one to six carbon atoms) and includes straight and branched chains. In an embodiment, C1-C6 alkyl groups are provided herein. Examples include methyl, ethyl, propyl, isopropyl, butyl, isobutyl, tert butyl, pentyl, neopentyl, and hexyl. Other examples of C1 C6-alkyl include ethyl, methyl, isopropyl, isobutyl, n-pentyl, and n-hexyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is partially or fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, C4-C7 cycloalkyl groups are provided herein.

As used herein, the term “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocycloalkyl also includes bicyclic structures that may be bridged or spirocyclic in nature with each individual ring within the bicycle varying from 3-8 atoms, and containing 0, 1, or 2 N, O, or S atoms. The term “heterocycloalkyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo[2.1.1]hexanyl, 6-azabicyclo[3.1.1]heptanyl, 2-azabicyclo[2.2.1]heptanyl, 3-azabicyclo[3.1.1]heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicyclo[3.2.1]octanyl. In an embodiment, C2-C7 heterocycloalkyl groups are provided herein.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms. In an embodiment, C5-C7 aryl groups are provided herein.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, C2-C7 heteroaryl groups are provided herein.

It is to be understood that if an aryl, heteroaryl, cycloalkyl, or heterocycloalkyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thioenyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The present disclosure provides inosine-5′-monophosphate dehydrogenase (IMPDH)-inhibiting nucleoside derivatives having anti-infective activities.

IMPDH is a purine biosynthetic enzyme which is highly conserved across all domains of life. As the de novo purine synthesis pathway is responsible for producing the bulk of guanine used for new RNA and DNA synthesis, the proper functioning of IMPDH is significant for the health of all rapidly proliferating biology, even viruses. By comparison, it appears that cells that are not actively dividing, such as most adult mammalian somatic cells, have relatively little demand for new nucleobases as they are less adversely affected by IMPDH inhibition (vide infera). For such cells, the only other metabolic source of guanine, the salvage pathway, may be sufficient. This difference in IMPDH dependency between slow and rapidly dividing cells provides a useful therapeutic index even for IMPDH inhibiting drugs that have relatively little to no selectivity for different IMPDH enzymes (vide infera).

The present disclosure provides a family of drugs that are either known nucleoside- and nucleotide-based IMPDH inhibitors, or are novel derivatives of such inhibitors, both of which have therapeutic anti-infective activity against certain viral, bacterial, fungal, and anti-protozoal infections.

The family of nucleoside and nucleotide IMPDH inhibitors are generally only active in their nucleotide form. This makes prodrugs the more commonly used nucleoside versions of these drugs, such as Ribavirin. These prodrugs that require activation in the form of net 5′-phosphorylation (see diagram below).

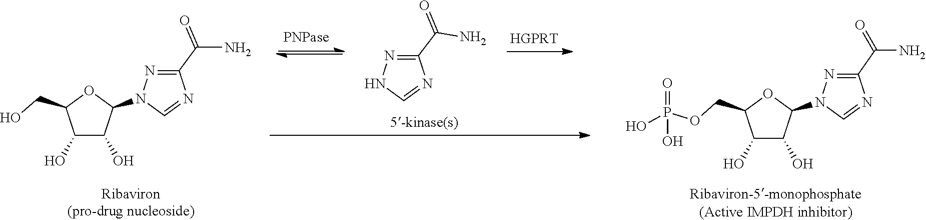 [SMILES: NC(=O)c1ncn([C@@H]2O[C@H](CO)[C@@H](O)[C@H]2O)n1 ; NC(=O)c1nc[nH]n1 ; NC(=O)c1ncn([C@@H]2O[C@H](COP(=O)(O)O)[C@@H](O)[C@H]2O)n1]

This net 5′-phosphorylation can be achieved in some organisms directly by kinases (bottom pathway in diagram), or can also be achieved by the sequential activity of purine nucleoside phosphorylase (PNPase) and then hypoxanthine-guanine phosphoribosyl-transferase (HGPRT) to transfer the base to a 5′-phosphorilated ribose (top pathway in diagram).

This activation of the nucleoside pro-drugs is in competition with degradation of the nucleoside and activated nucleotide species (see diagram below.)

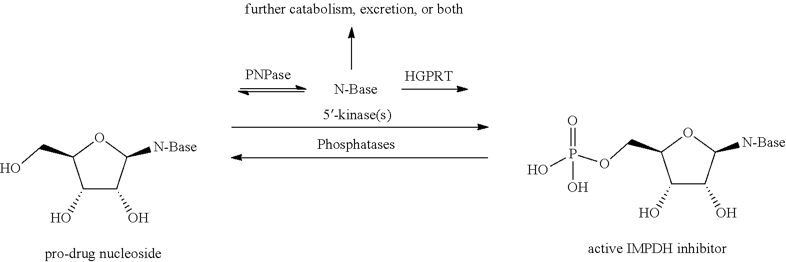

The active nucleotide form of these drugs can be deactivated by phosphatases, typically 5′-nucleotidases, to yield the inactive nucleoside species. Similarly, while PNPase can be the first step in activating these nucleoside-based drugs, this initial step is catabolic. This catabolic process leaves the free nucleobase exposed to any combination of further and irreversible catabolic breakdown steps as well as excretion. All of these alternatives are in competition with the necessary activation by HGPRT.

This flux of competing activation and deactivation/degradation processes results in multiple draw backs to traditional members of the family of nucleoside and nucleotide IMPDH inhibitors as far as their use as drugs. First, it means that in vivo at least some portion of the dosing of these drugs are always in the various therapeutically-inactive states that are part of this flux. Thus, the effective active drug concentration must always be lower than what was dosed. Second this necessarily means that there are multiple unnatural drug-derived products present, and this inherently increases the risk of the drug being “dirty”—i.e. have off-target binding that increases side effects and often decreases the therapeutic index as well. Third, this often means somewhere within this metabolic flux there is an existing highly efficient pathway to degrade these compounds potentially limiting the desired PK/PD profiles, or in relation to the nature of this patent proposal: allowing infective agents to easily detoxify these potential drugs via some combination of efficient degradation and excretion (efflux).

The nucleoside analogs according to the disclosure are taken up more easily and accumulate in greater amounts in certain infective agents than are their corresponding nucleotide or nucleoside. Also, these derivatives require no activation to a nucleotide species, thereby allowing a less restrictive range of nucleobases to be used, as they do not need to be recognized by an appropriate kinase or equivalent set of enzymes in addition to the activated drug's ultimate IMPDH target. In addition, these derivatives are not inactivated by phosphatases. They are not part of the standard metabolic flux of nucleosides and nucleotides that is catabolized by PNPase and thereby exposed to additional processes for irreversible nucleobase catabolism. As a result, these drugs exist with a higher fraction in their active form at any given time in vivo, and degrade more slowly over time relative to their equivalent nucleoside- or nucleotide-based IMPDH inhibitors. Accordingly, these derivatives require a lowered dosing to achieve the same levels of effect, and demonstrate cleaner and safer drug profiles then traditional nucleoside and nucleotide based IMPDH inhibitors.

### IMPDH-Inhibiting Nucleoside Derivatives

Useful IMPDH-inhibiting nucleoside derivatives fall into the generic structure of

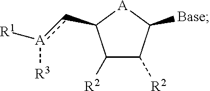

or a pharmaceutically acceptable salt thereof, wherein:
Base is selected from the group consisting of

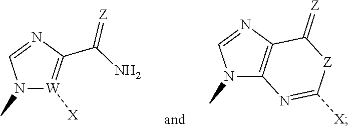

A is selected from the group consisting of —CH—, —CH₂—, —N—, —NH—, —O—, —SO₂R³—, and —S—;
W is selected from the group consisting of —C—, —CH—, —CH₂—, —N—, and —NH₂—;
X is, independently at each occurrence, selected from the group consisting —OH, —SH, —NH₂ halogen;
Y is selected from the group consisting of —OH, —SH, —NH₂, and —N₃;
Z is selected from the group consisting of O, S, and;
R¹ is selected from the group consisting of —PA₁O₂(R³)₂, — and —SO₂R³;
R² is, independently at each occurrence, selected from the group consisting of —OH, —NH₂, and —N₃;
R³ is, independently at each occurrence, selected from the group consisting of —H, —C₁₋₆ alkyl, —C₁₋₆ alkenyl, —C₁₋₆ allyl —C₆₋₁₀ aryl, and —N(R⁴)₂, wherein —C₁₋₆ alkyl is optionally substituted with one or more halo;
R⁴ is, independently at each occurrence, selected from the group consisting of —H, —C₁₋₆ alkyl, —C₁₋₆ alkenyl, —C₁₋₆ allyl and —C₆₋₁₀ aryl; and

[symbol] is optional bond, and

wherein the derivative is not

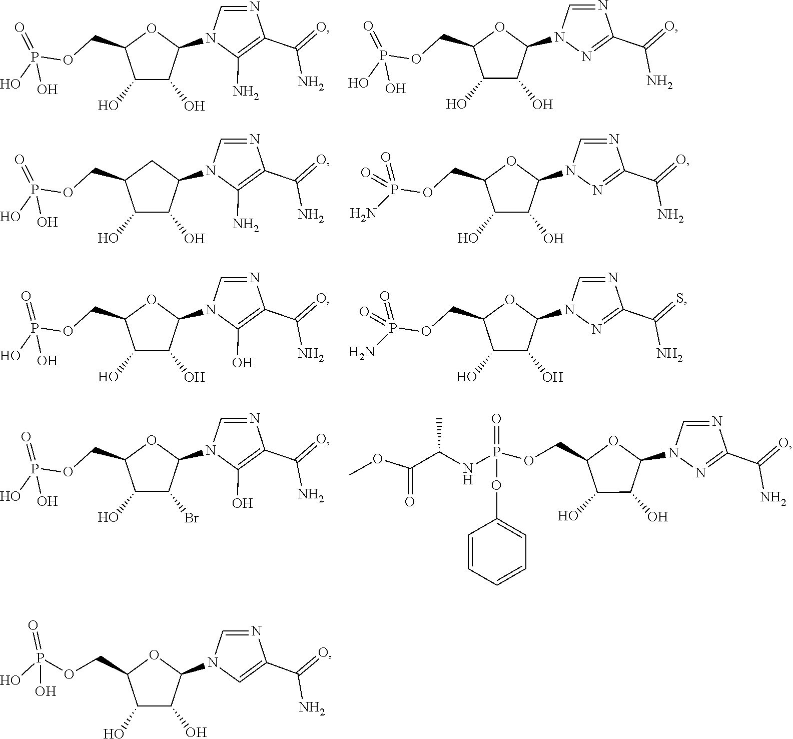

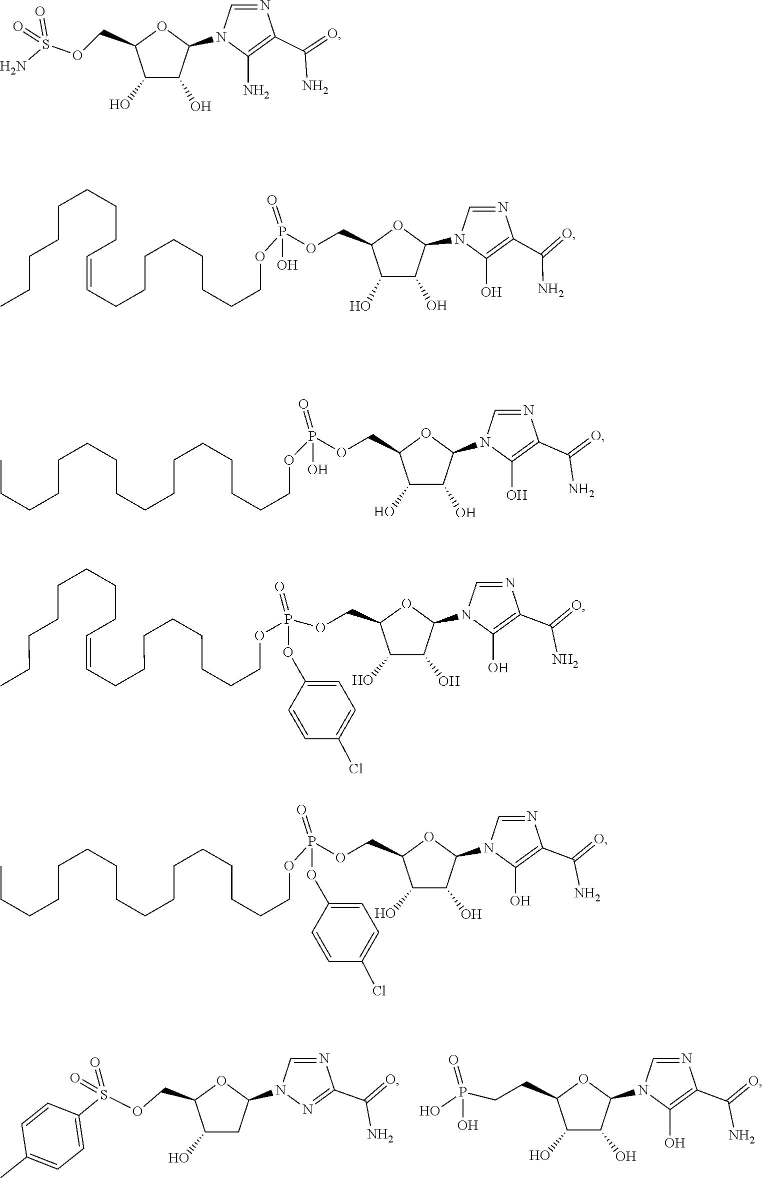

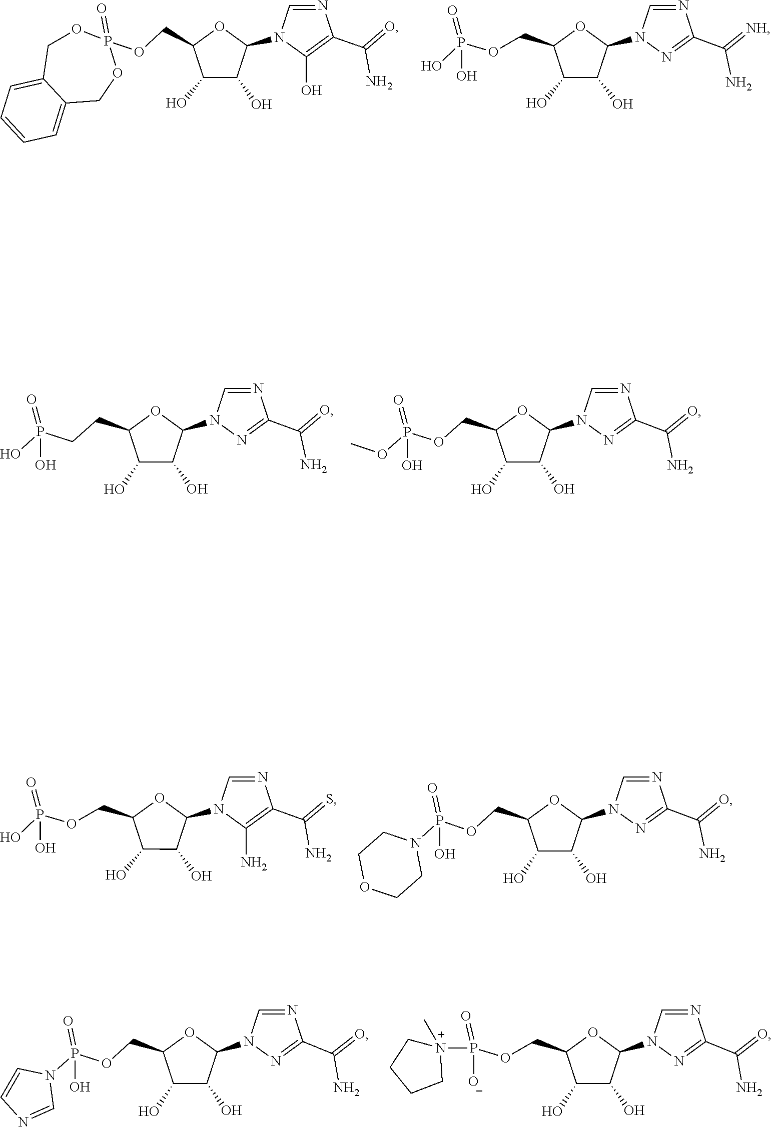

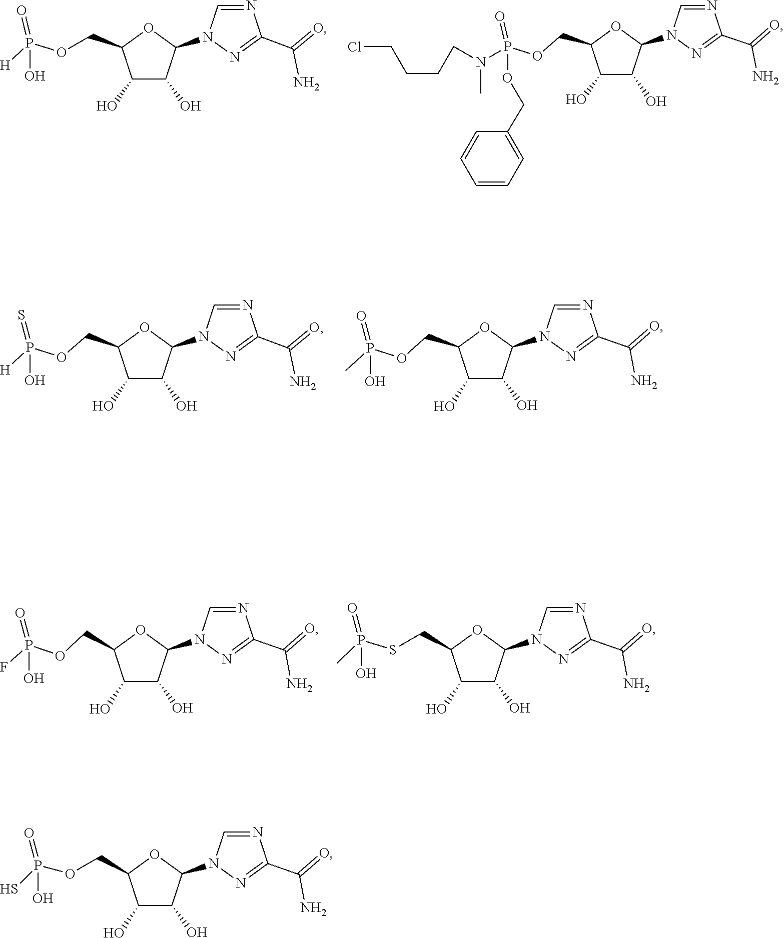

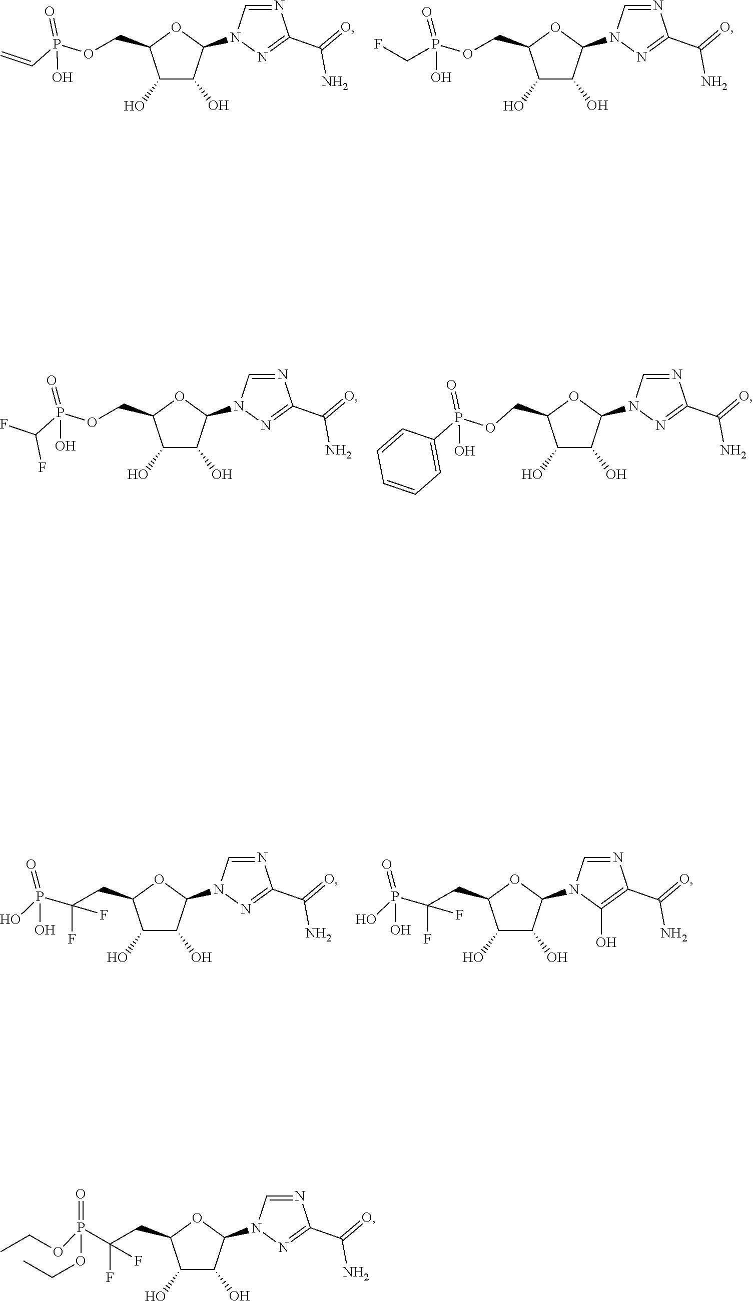

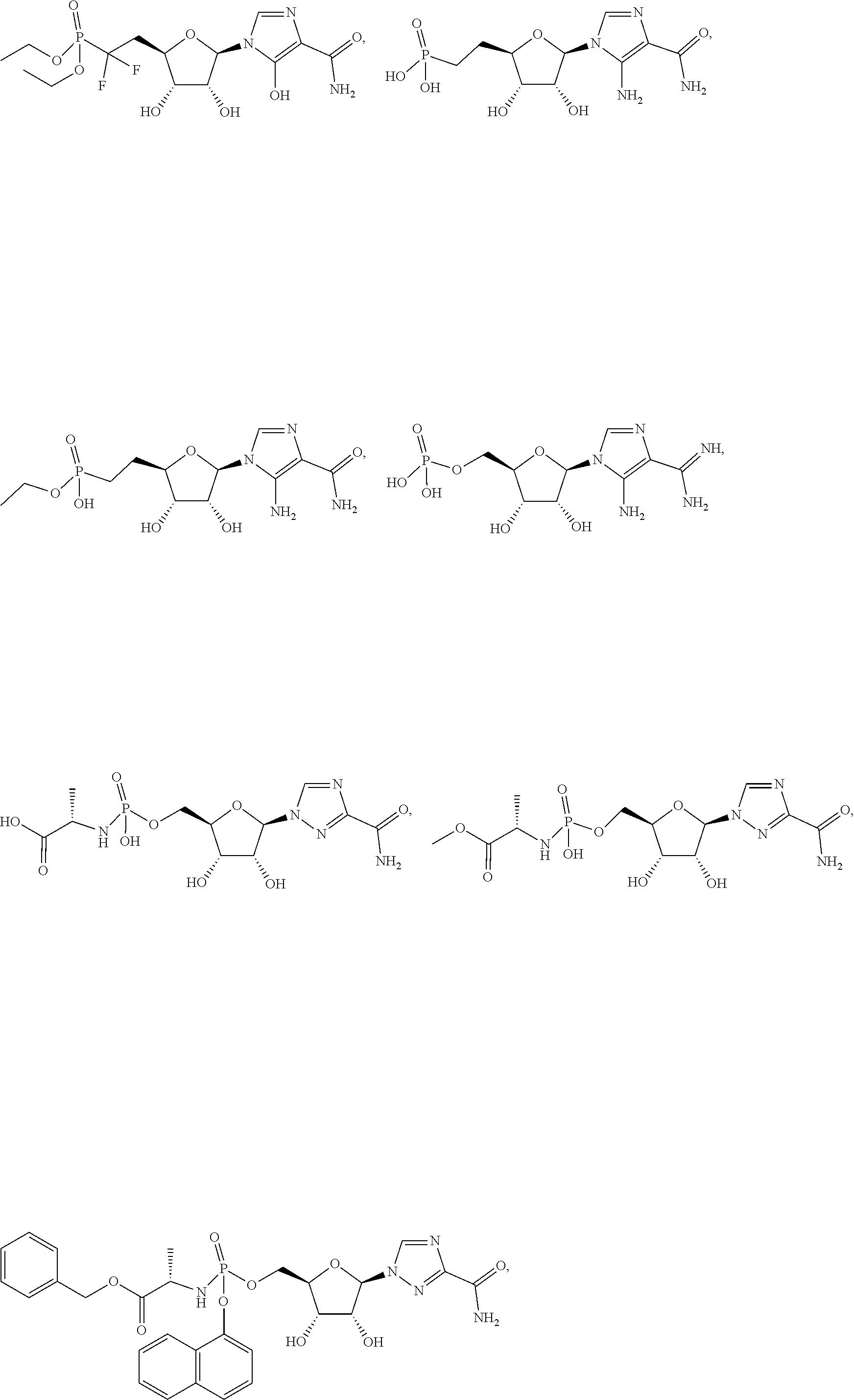

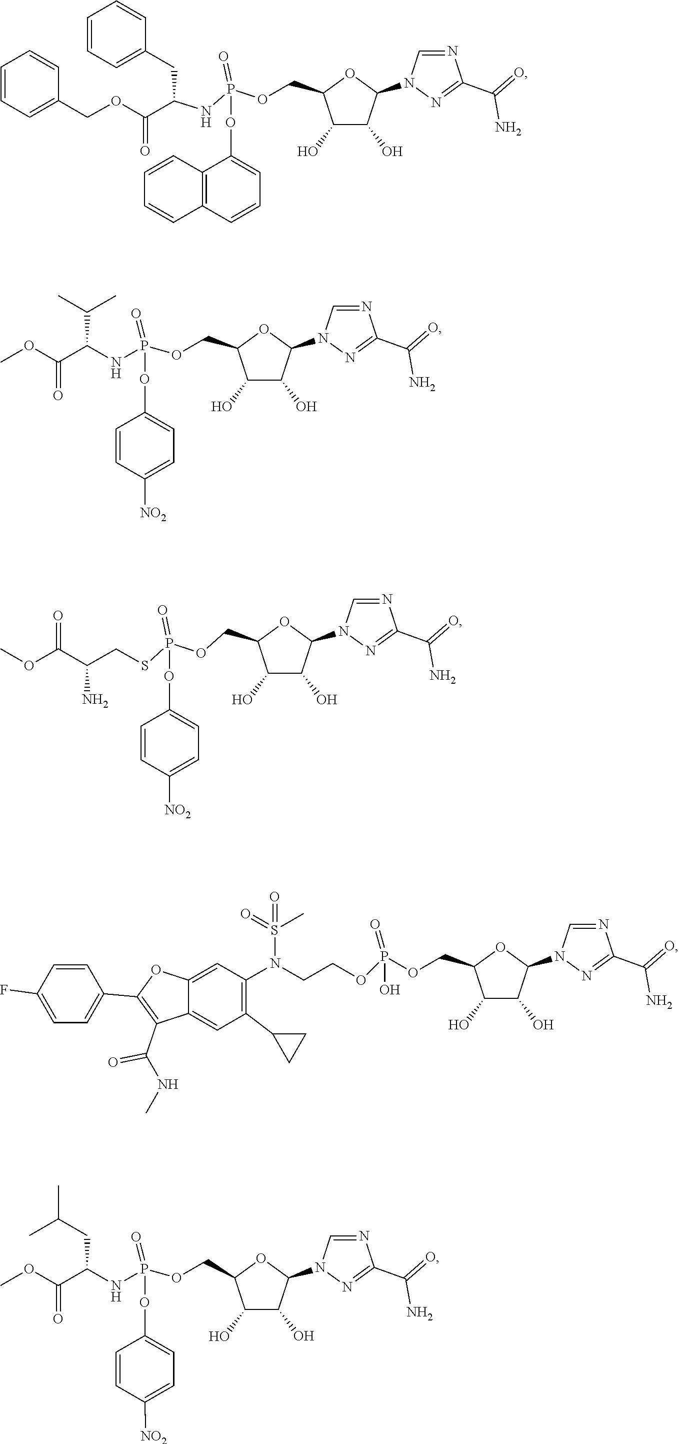

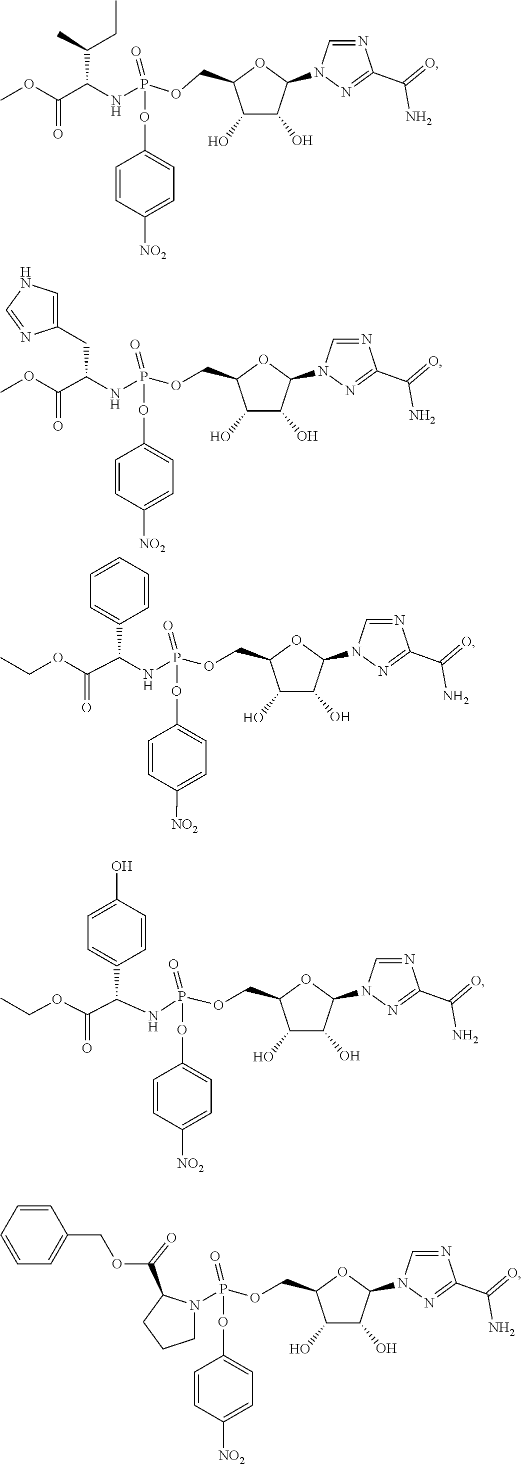

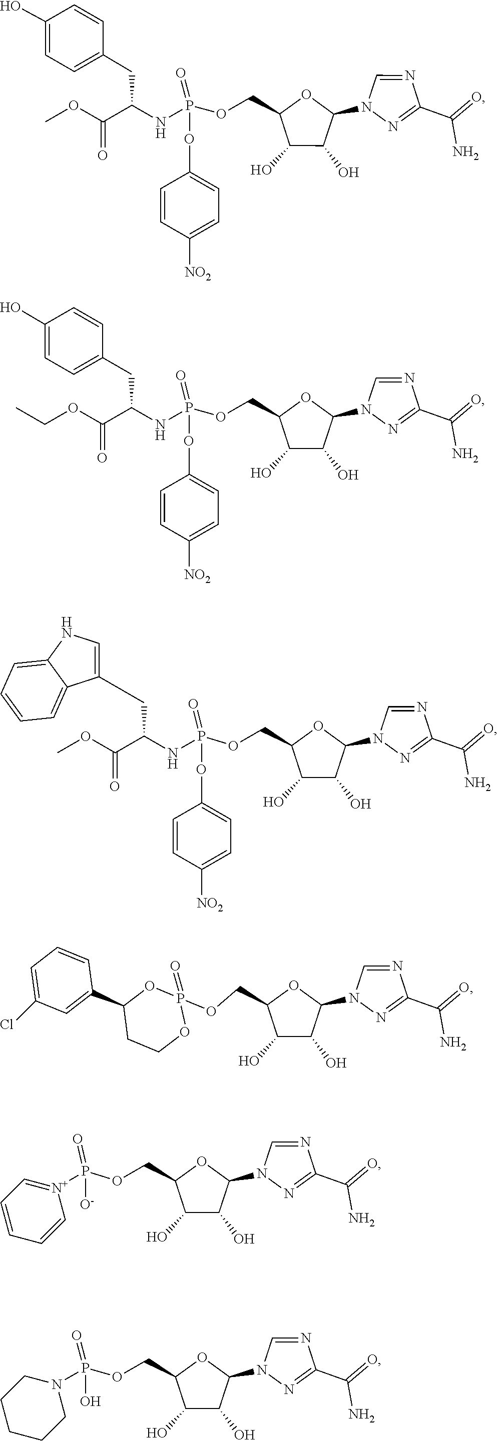

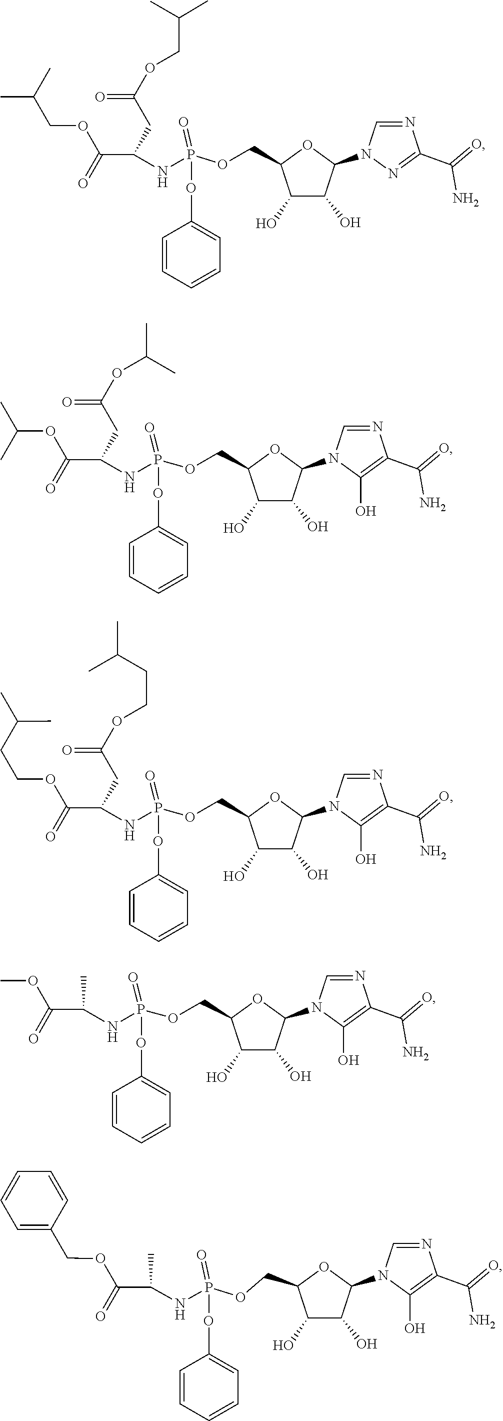

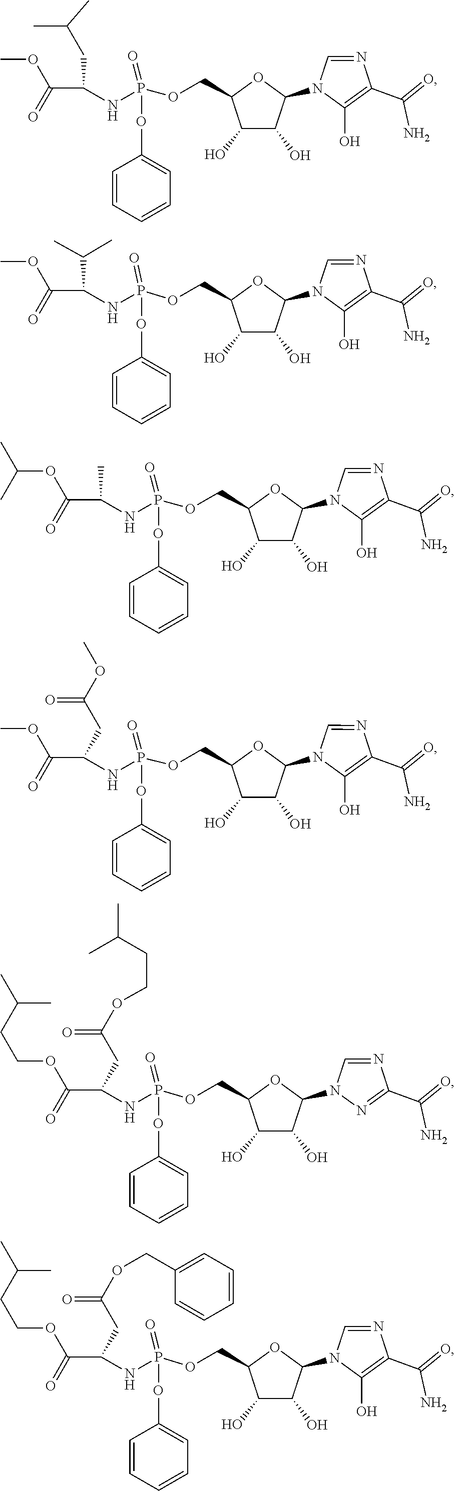

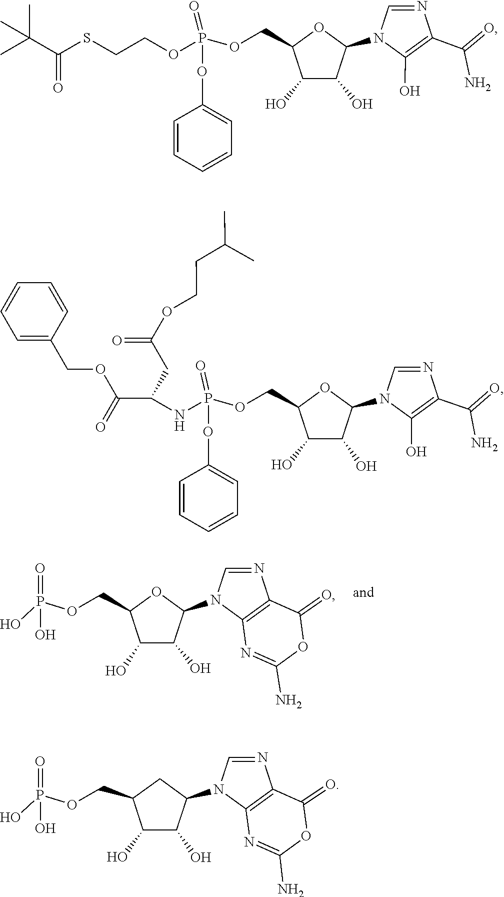

In yet another embodiment, the nucleoside analog inhibitor is a compound of Formula II:

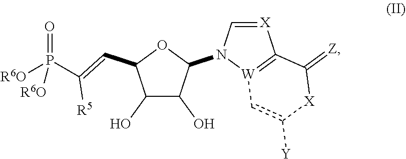

a compound of Formula III:

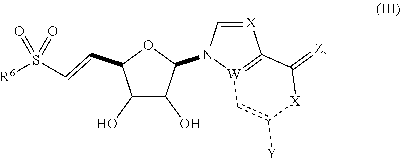

a compound of Formula IV:

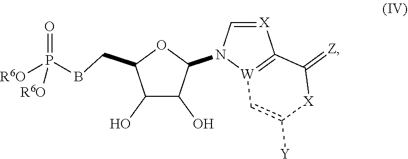

a compound of Formula V:

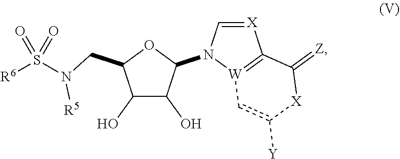

or a pharmaceutically acceptable salt of a compound of Formula II, Formula III, Formula IV, or Formula V, wherein:
W is selected from the group consisting of —C—, —CH₂—, —N—, and —NH₂—;
X is, independently at each occurrence, selected from the group consisting of —O—, —OH, —S—, —SH, —NH—, —NH₂, —CH₂—, and —CH₃;
Y is selected from the group consisting of —OH, —SH, —NH₂, and —N₃;
Z is selected from the group consisting of O, S, and NH;
B is selected from the group consisting of S, O, NH, and NR⁵;
R⁵ is selected from the group consisting of —H, halo, —C₁₋₆ alkyl, —C₁₋₆ alkenyl, and —C₆₋₁₀ aryl;
R⁶ is, independently at each occurrence, selected from the group consisting of —H, —C₁₋₆ alkyl, —C₁₋₆ alkenyl, —C₆₋₁₀ aryl, —CH₂—C₆₋₁₀ aryl, —O—C₆₋₁₀ aryl, —N(C₁₋₆ alkyl)₂, —NH(C₁₋₆ alkyl), and —NH₂, wherein —C₆₋₁₀ aryl is optionally substituted with one or more R⁷;
or alternatively, each R⁶, together with the atom to which they are attached, can form C₃₋₁₂ heterocycle or C₃₋₁₂ heteroaryl, wherein heterocycle or heteroaryl is optionally substituted with one or more R⁷;
R⁷ is selected from the group consisting of halo, —C₁₋₆ alkyl, —C₁₋₆ alkenyl, —C₆₋₁₀ aryl, —OC₁₋₆ alkyl, —N(C₁₋₆ alkyl)₂, —NH(C₁₋₆ alkyl), —NH₂, and —OH; and
[symbol] is an optional bond.

In specific embodiments, the compounds of Formulae I-N are compounds having one of the following structures:

| Structure | |
|---|---|
| No. | Structure |
| 1 | 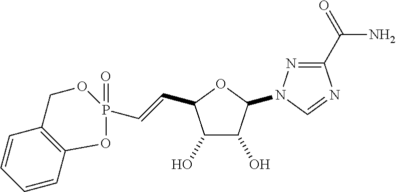 [SMILES: NC(=O)c1ncn([C@@H]2O[C@H](/C=C/P3(=O)OCc4ccccc4O3)[C@@H](O)[C@H]2O)n1] |
| 2 | 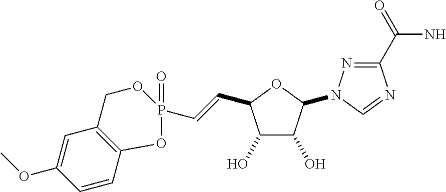 [SMILES: COc1ccc2c(c1)COP(=O)(/C=C/[C@H]1O[C@@H](n3cnc(C(N)=O)n3)[C@H](O)[C@@H]1O)O2] |
| 3 | 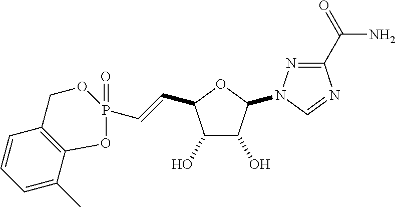 [SMILES: Cc1cccc2c1OP(=O)(/C=C/[C@H]1O[C@@H](n3cnc(C(N)=O)n3)[C@H](O)[C@@H]1O)OC2] |
| 4 | 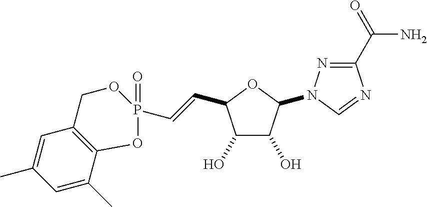 [SMILES: Cc1cc(C)c2c(c1)COP(=O)(/C=C/[C@H]1O[C@@H](n3cnc(C(N)=O)n3)[C@H](O)[C@@H]1O)O2] |
| 5 | 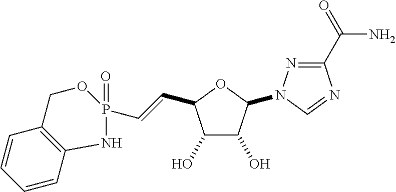 [SMILES: NC(=O)c1ncn([C@@H]2O[C@H](/C=C/P3(=O)Nc4ccccc4CO3)[C@@H](O)[C@H]2O)n1] |
| 6 | 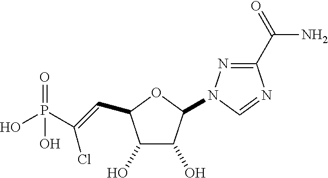 |
| 7 | 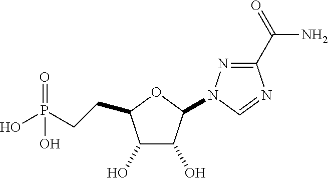 |
| 8 | 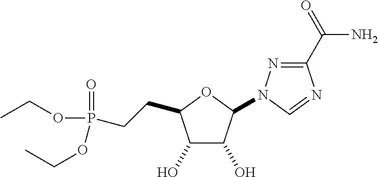 |
| 9 | 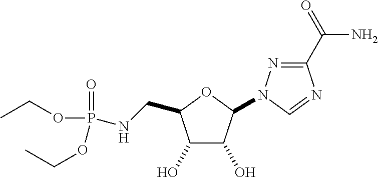 |
| 10 | 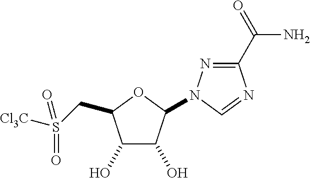 |
| 11 | 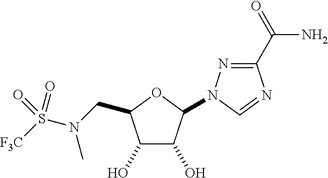 |
| 12 | 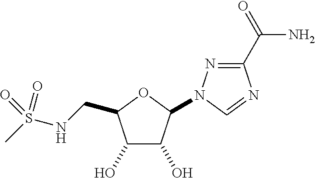 |
| 13 | 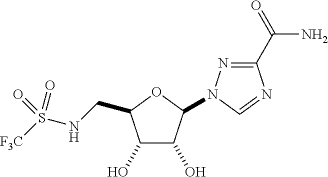 |
| 14 | 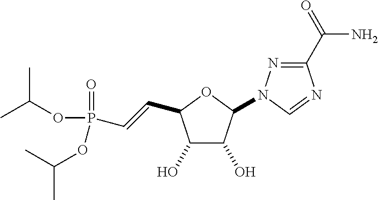 |
| 15 | 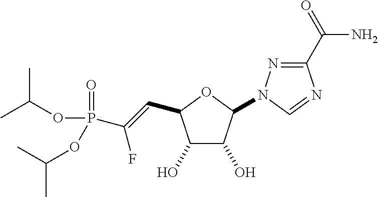 |
| 16 | 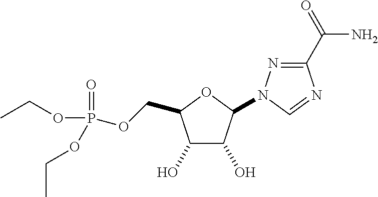 |
| 17 | 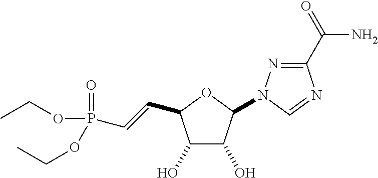 |
| 18 | 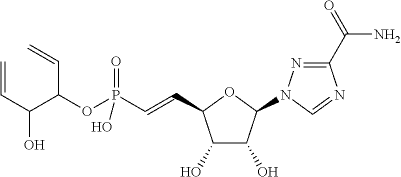 [SMILES: C=CC(O)C(C=C)OP(=O)(O)/C=C/[C@H]1O[C@@H](n2cnc(C(N)=O)n2)[C@H](O)[C@@H]1O] |
| 19 | 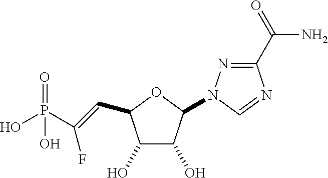 |
| 20 | 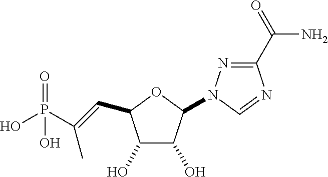 |
| 21 | 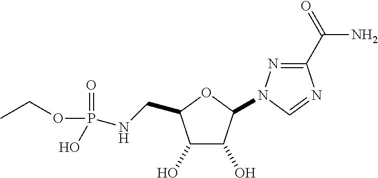 |
| 22 | 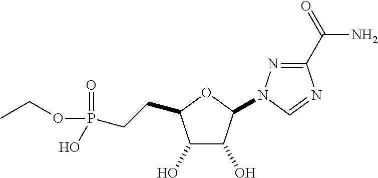 |
| 23 | 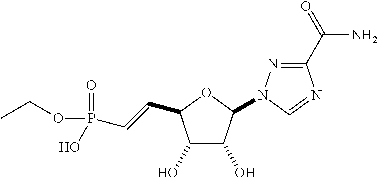 |
| 24 | 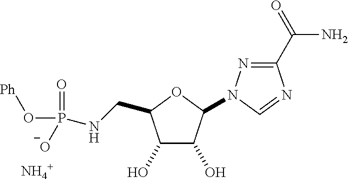 |
| 25 | 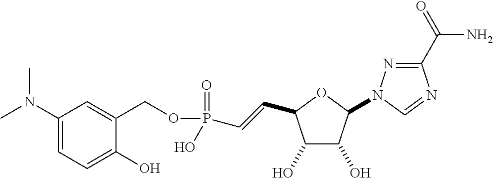 |
| 26 | 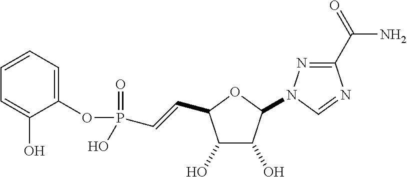 |
| 27 | 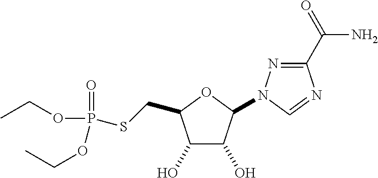 |
| 28 | 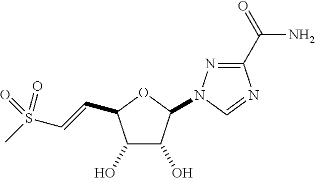 |
| 29 | 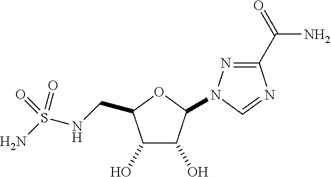 |
| 30 | 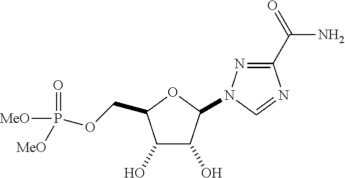 |
| 31 | 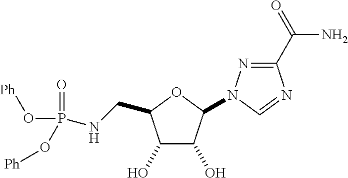 |
| 32 | 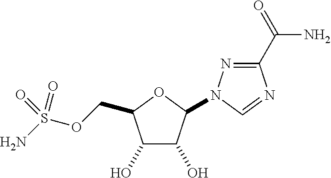 |
| 33 | 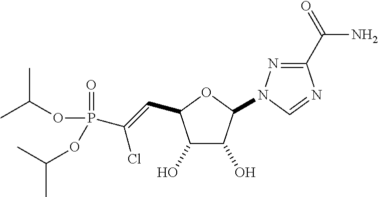 |
| 34 | 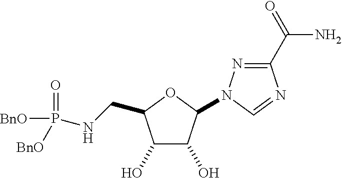 |
| 35 | 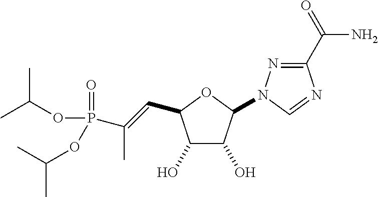 |
| 36 | 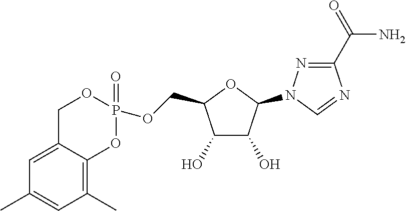 [SMILES: Cc1cc(C)c2c(c1)COP(=O)(OC[C@H]1O[C@@H](n3cnc(C(N)=O)n3)[C@H](O)[C@@H]1O)O2] |

or a pharmaceutically acceptable salt thereof.

### Syntheses of Nucleoside Derivatives

The nucleoside derivatives useful in the formulations for treating an infection can wither be commercially obtained or can be synthesized by any method known in the art. Representative methods for the different classes of 5′ derivatives are provided below.

From these symmetric alkyl phosphonite esters, the free phosphate is afforded by the use of TMSBr or an equivalent oxophilic Lewis acid (e.g., TMSOtf, etc.). More complex alkyl phosphonate esters, non-alkyl phosphonate esters and amidates (e.g. R¹ and R²=aryl, or allyl), non-symmetrical phosphonate esters and amidates (e.g., R¹≠R²), and mono esters or amidates (e.g., either R¹═H while R²=alkyl, allyl, or aryl), are then produced by dehydrative coupling, via DCC, CDI, or equivalent, with the corresponding R¹ and R² alcohols or amines. The acetal-free products of any of these species are then yielded via hydrolysis with a protic acid (e.g., TFA in water).

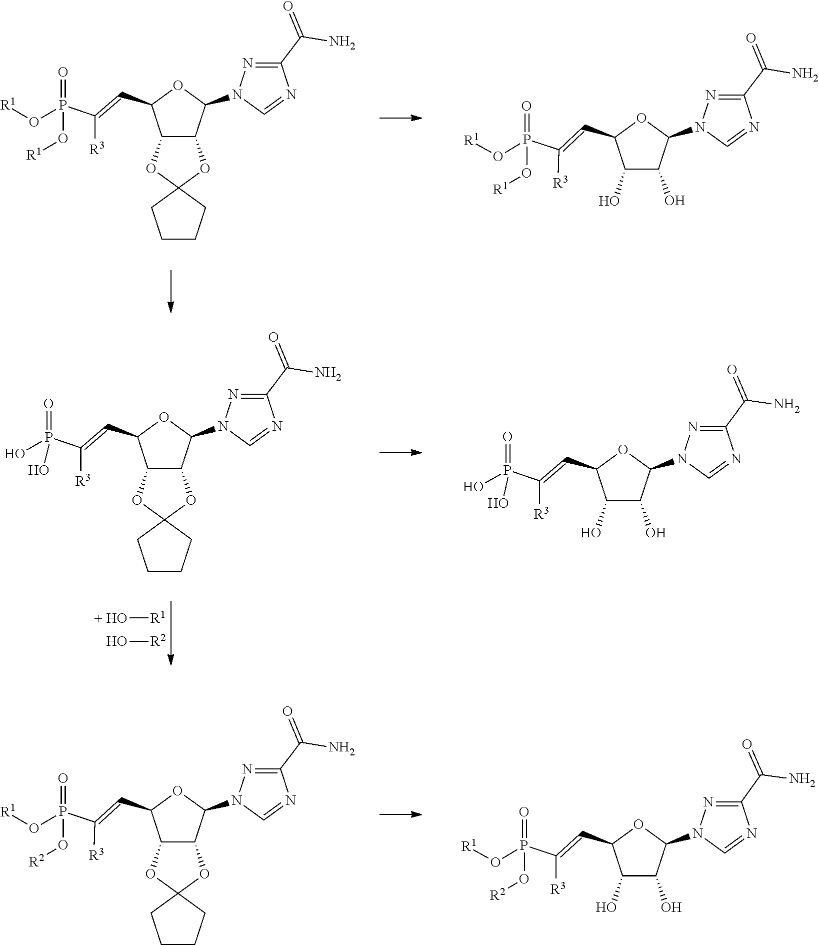

Representative vinyl—phosphonate derivatives that can be synthesized by this route include:

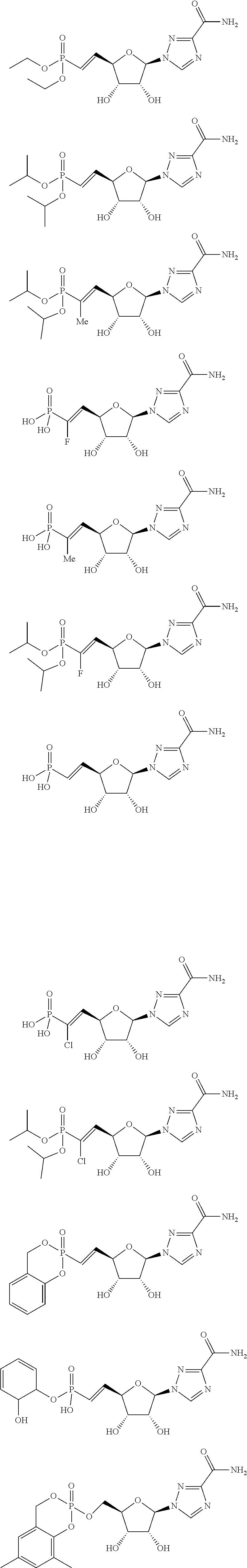

### 2. Synthesis of Vinyl-Sulphone, Vinyl-Sulfonyl, and Vinyl-Sulfonamides

Vinyl-sulphone, vinyl-sulfonyl, and vinyl-sulfonamides may be produced from the same aldehyde intermediate that the vinyl-phosphonate species are prepared from. An equivalent Homer-Wadsworth-Emmons reaction (ibid.) is performed with a methyl phosphonate of the corresponding sulphone, sulfonyl, or sulfonamide. The final product is afforded by cleaving the acetal under acidic conditions.

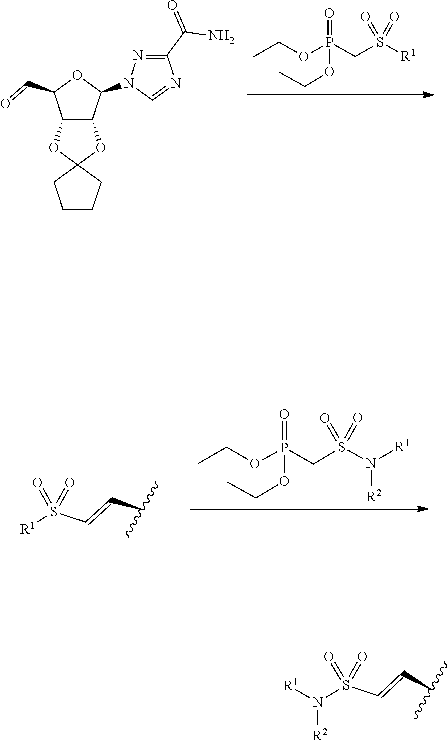

A representative vinyl-sulfone derivative that can be synthesized by this route is:

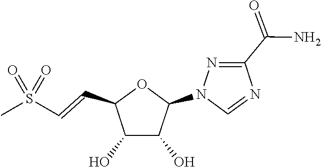 [SMILES: CS(=O)(=O)/C=C/[C@H]1O[C@@H](n2cnc(C(N)=O)n2)[C@H](O)[C@@H]1O]

### 3. Synthesis of Phosphoramidates, N-Bound-Sulfonamides, and N-Bound-Sulfamides

Phosphoramidates, N-bound-sulfonamides, and N-bound-sulfamides on secondary nitrogens are produced through the synthesis of 5′-amino 2′,3′-acetonide of the base nucleoside (so far, ribavirin). Commercially available nucleoside (e.g., ribavirin) is converted into 2′,3′-acetal under acidic conditions via the corresponding ketone—cyclohexanone, but alternatively, acetone may be used. The 5′-alcohol on the resulting species is converted into a leaving group, such as tosylate. The leaving group is displaced with azide, and in a separate step the azide is reduced down into an amine by any number of means (e.g., hydrogenation with palladium on carbon).

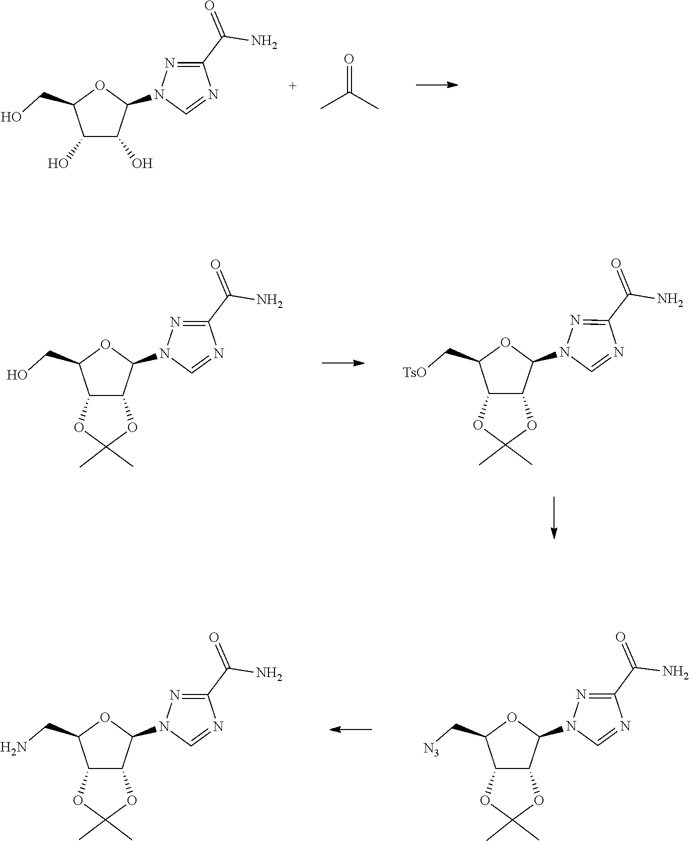

Phosphoramidates with two esters (i.e., both R¹ and R²≠H) are prepared from this common 5′-amine intermediate via reaction with either the corresponding phosphoryl chloride (top of arrow) or phosphite (bottom of arrow) species, which in turn are obtained via existing literature preps or commercially. Mono-ester phosphoramidates are prepared by partial hydrolysis of the appropriate di-ester phosphoramidates with either base (e.g., NH3, NaOH, etc.) or oxophilic Lewis acids (e.g., TMS-Br.) For both phosphoramidate mono-esters and di-esters the final product is yielded via acid catalyzed hydrolysis of the acetal.

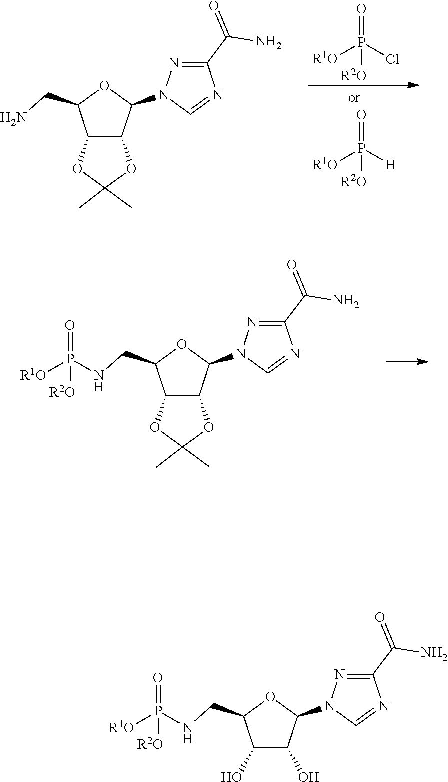

Representative phosphoramidate derivatives that can be synthesized by this route include:

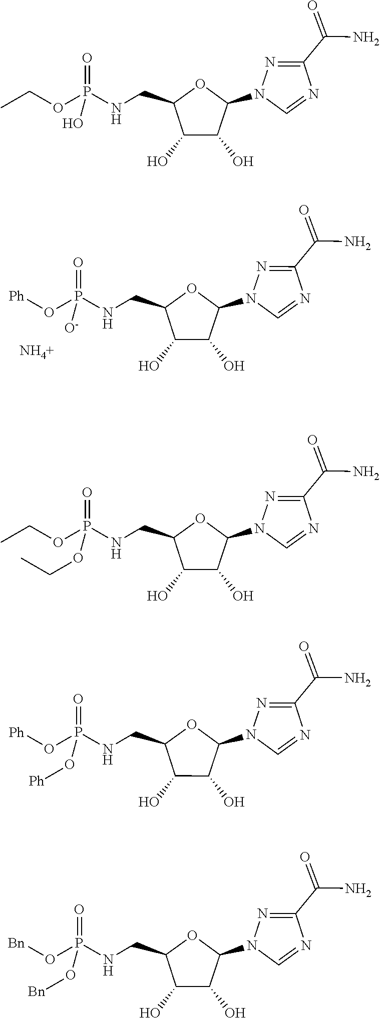 [SMILES: CCOP(=O)(O)NC[C@H]1O[C@@H](n2cnc(C(N)=O)n2)[C@H](O)[C@@H]1O ; NC(=O)c1ncn([C@@H]2O[C@H](CNP(=O)([O-])Oc3ccccc3)[C@@H](O)[C@H]2O)n1 ; [NH4+] ; CCOP(=O)(NC[C@H]1O[C@@H](n2cnc(C(N)=O)n2)[C@H](O)[C@@H]1O)OCC ; NC(=O)c1ncn([C@@H]2O[C@H](CNP(=O)(Oc3ccccc3)Oc3ccccc3)[C@@H](O)[C@H]2O)n1 ; NC(=O)c1ncn([C@@H]2O[C@H](CNP(=O)(OCc3ccccc3)OCc3ccccc3)[C@@H](O)[C@H]2O)n1]

The N-bound-sulfonamides and N-bound-sulfamides on secondary nitrogens are produced from the common 5′-amino 2′,3′-acetal-protected nucleoside intermediate with the corresponding sulfur based reagents, which in turn are commercially available or can be prepared by any method known in the art. The final product is again yielded via acid catalyzed hydrolysis of the 2′,3′ acetal.

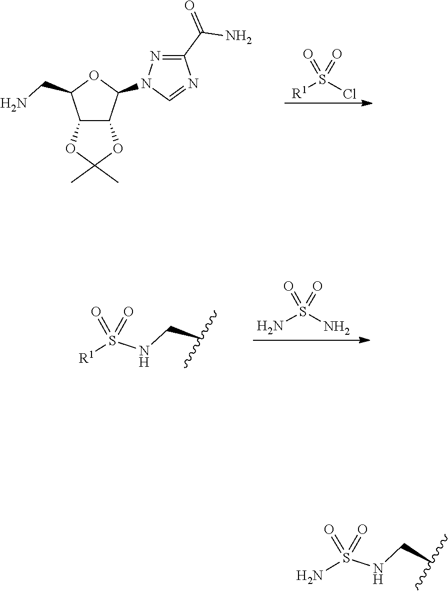

Representative N-bound-sulfonamide, and -sulfamide derivatives that may be synthesized by this route include:

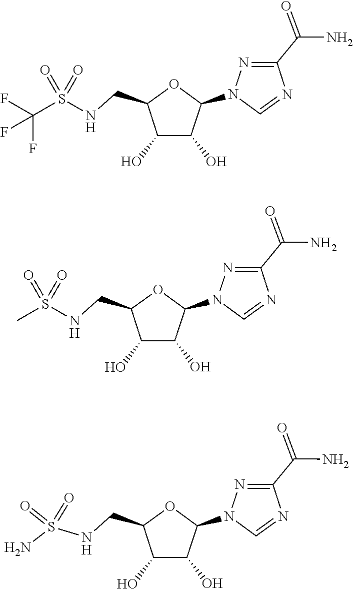 [SMILES: NC(=O)c1ncn([C@@H]2O[C@H](CNS(=O)(=O)C(F)(F)F)[C@@H](O)[C@H]2O)n1 ; CS(=O)(=O)NC[C@H]1O[C@@H](n2cnc(C(N)=O)n2)[C@H](O)[C@@H]1O ; NC(=O)c1ncn([C@@H]2O[C@H](CNS(N)(=O)=O)[C@@H](O)[C@H]2O)n1]

### 4. Synthesis of N-substituted, N-bound Sulfonamide

N-substituted, N-bound-sulfonamides on tertiary nitrogens are produced through the synthesis of an N-substituted 5′-amino 2′,3′-acetonide of the base nucleoside. Commercially available nucleoside (e.g., ribavirin) is converted into 2′,3′-acetal under acidic conditions via the corresponding ketone—cyclohexanone preferred, but alternatively, acetone can be used. The 5′-alcohol on the resulting species is converted into a leaving group, such as tosylate. The leaving group is directly displaced the desired mono substituted amine, which in turn is obtained via existing literature preps or commercially. The appropriate sulfonating reagent is used to convert the 5′ amine into the desired fully substituted sulfonamide. Acid catalyzed hydrolysis of the acetal yields the final product.

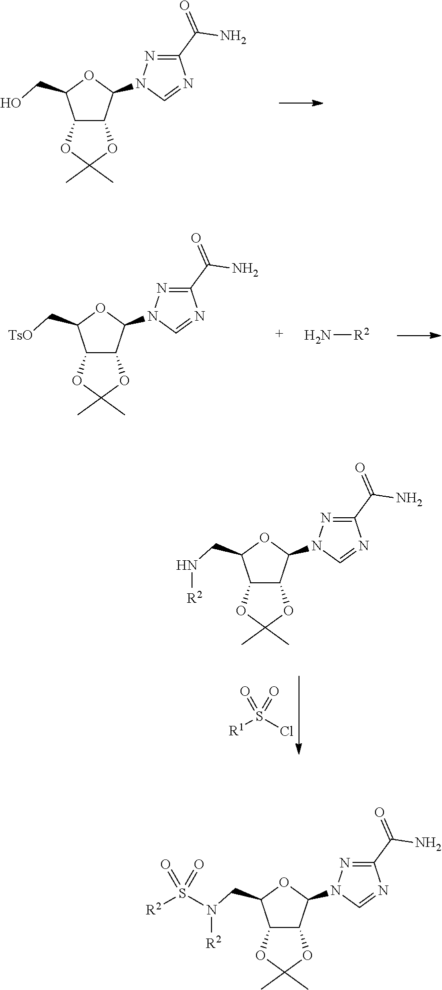

A representative N-substituted, N-bound-sulfonamide derivative that may be synthesized by this route is:

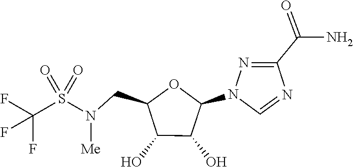 [SMILES: CN(C[C@H]1O[C@@H](n2cnc(C(N)=O)n2)[C@H](O)[C@@H]1O)S(=O)(=O)C(F)(F)F]

Infections Treatable with the Nucleoside Analog Derivatives

The inhibitory effects against central metabolism of a number of problematic infective organisms enable the known and novel derivatives according to the disclosure to treat infections and indications resulting from the infection of various organisms. Such infective organisms, the growth of which can be inhibited by the derivatives according to the disclosure include, but are not limited to, Gram negative bacteria including, but are not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, and Bacteroides splanchnicus.

These derivatives can also treat the infection of Gram positive pathogenic bacteria including, but not limited to, Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, Staphylococcus saccharolyticus, Clostridium difficile, Clostridium perfringens, Clostridium tetini, and Clostridium botulinum.

Other types of bacterial infections can also be treated with the derivatives according to the disclosure include, but not limited to, Mycobacterium tuberculosis, Mycobacterium avium, Mycobacterium intracellulare, and Mycobacterium leprae.

Additionally, the derivatives according to the disclosure can treat non-bacterial infections of yeast, fungi, and multicellular parasites. Exemplary types of infection to which formulations comprising a derivative according to the disclosure can be applied, include, but are not limited to, respiratory, pulmonary, and other compartments including, but not limited to the skin, rectum, vagina, urethra, urinary tract, bladder, eye, and ear.

### Pharmaceutical Formulations and Treatment

The pharmaceutical formulations useful in the therapeutic methods according to the disclosure include a therapeutically effective amount of a derivative according to the disclosure which has anti-infective properties, and which is not heretofore known to have anti-infective properties, in a pharmaceutically acceptable carrier. A “therapeutically effective amount” as used herein refers to that amount of the derivative which provides a therapeutic and/or prophylactic therapeutic effect for treating, reducing the symptoms of, or inhibiting the progression of, an infection of a problematic organism.

The pharmaceutical formulations according to the disclosure further comprise a pharmaceutically acceptable carrier. The term “pharmaceutically acceptable carrier” is to be understood herein as referring to any substance that may, medically, be acceptably administered to a patient, together with ta derivative according to the disclosure, and which does not undesirably affect the pharmacological and synergistic activity of the compound. A “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington, The Science and Practice of Pharmacy (9th Ed. 1995).

For use in medicine, the salts of the anti-infective compounds are pharmaceutically acceptable salts. Other salts may, however, be useful in the preparation of the compounds or of their pharmaceutically acceptable salts according to the disclosure. Suitable pharmaceutical-salts of the compounds according to the present disclosure include acid addition salts which may, for example, be formed by mixing a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Additionally, where at least one of the anti-infective compounds in the combination formulation disclosure carry an acidic moiety, suitable pharmaceutically acceptable salts thereof may include alkali metal salts, e.g., sodium or potassium salts; alkaline earth metal salts, e.g. calcium or magnesium salts; and salts formed with suitable organic ligands, e.g. quaternary ammonium salts.

The pharmaceutical formulation may be prepared for injectable use, topical use, oral use, intramuscular or intravenous injection, inhalation use, transdermal use, intradermal, transmembrane use, and the like

These formulations are in unit dosage forms such as tablets, pills, capsules, powders, granules, sterile parenteral solutions or suspensions, metered aerosol or liquid (nebulized) sprays, drops, ampoules, auto-injector devices or suppositories; for oral parenteral, intranasal, sublingual topical or rectal administration, or for administration by inhalation or insufflation. Alternatively, the formulations may be presented in a form suitable for one-weekly or once-monthly administration; for example, an insoluble salt of the derivative, such as decanoate salt, may be adapted to provide a depot preparation for intramuscular injection. An erodible polymer containing the derivative may be envisaged.

The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as, but no limited to, ether, ethyl acetate, ethanol, isopropanol, or acetonitrile are useful. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66: 2 (1977).

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a pharmaceutically acceptable salt thereof.

These formulations may be homogeneous, i.e., the derivative is dispersed evenly throughout the composition so that the composition may be readily subdivided into equally effective unit dosage forms such as tablets, pills and capsules. This solid formulation composition is then subdivided into unit dosage forms of the type described above containing from about 0.1 mg to about 500 mg of the derivative of the present disclosure. Some useful unit dosage forms contain from 1 mg to 100 mg, for example, about 1 mg, about 2 mg, about 5 mg, about 10 mg, about 25 mg, about 50 mg, or about 100 mg, of the derivative. The tablets or pills of the novel composition can be coated or otherwise compounded to provide a dosage form affording the advantage of prolonged action. The liquid forms in which the novel derivatives of the present disclosure may be incorporated for administration orally or by injection include aqueous solutions, suitably flavored syrups, aqueous or oil suspensions, and flavored emulsions with edible oils as well as elixirs and similar pharmaceutical vehicles. In the treatment of infections, a suitable dosage level is about 0.001 mg/kg to about 250 mg/kg per day. The formulation may be administered on bolus and or a regimen of about 1 to 4 times per day.

Injectable dosage forms may be sterilized in a pharmaceutically acceptable fashion, for example by steam sterilization of an aqueous solution sealed in a vial under an inert gas atmosphere at 120° C. for about 15 minutes to 20 minutes, or by sterile filtration of a solution through a 0.2 μM or smaller pore-size filter, optionally followed by a lyophilization step, or by irradiation of a composition containing a compound of the present disclosure by means of emissions from a radionuclide source.

A therapeutically effective dosage of the formulation according to the disclosure may vary from patient to patient, and may depend upon factors such as the age and physical size of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 0.1 mg/kg to about 1000 mg/kg, or from about 1 mg/kg to about 100 mg/kg may be suitable

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Preliminary Screens for Anti-Infective IMPDH-Inhibiting Nucleoside Derivatives

To identify potential anti-infective nucleoside derivatives according to the disclosure, MHB (Millipore-Sigma, Burlington, Mass.) containing different concentrations of various nucleoside derivatives was inoculated with about 10⁶ CFU/mL of PA14. The screen was conducted by measuring growth of PA14bacteria under conditions: with MHB and with MHB+derivative. Bacterial growth was assessed by measurement of turbidity (OD₆₀₀) after incubation at 37° C. for 20 hr.

### Example 2

### In Vivo Testing of Compounds Via the Murine Thigh Model

### Pre-Treatment

Female CD-1 mice, 5-6 weeks old, (18-22 g) (Harlan Laboratories, Indianapolis, Ind.) are made neutropenic by administration of cyclophosphamide (Sigma-Aldrich, St. Louis, Mo.) on Days −4 (150 mg/kg) and −1 (100 mg/kg) of infection. Inoculum Preparation

On Day 0, animals were inoculated intramuscularly (0.1 ml/thigh) with ˜1×10⁵ CFU/mouse of the infective organism. In one representative study, the organisms used are bacteria (P. aeruginosa UNT202-1 (PA14), A. baumannii UNT190-1, E. coli UNT156-1, isolates and are part of the University of North Texas Health Sciences Center (Fort Worth, Tex.) culture collection). Inoculation is into the right thigh. One group did not receive drug treatment and their thighs were harvested at 1-hour post-infection. The remaining mice were administered test derivatives at standard times and route.

### Dose Preparation

Each test derivative was formulated by dissolving the compounds in either PBS or DMSO.

For each of the dose groups in the maximum tolerated dose (MTD) determination study, three (3) animals were used for each dose level. The use of three animals was sufficient for the determination of the MTD and this group size and proceeding in an ascending stepwise manner allowed for the use of as few animals as possible. Survival and general observations (breathing, mobility, reactions) as to the tolerability of the administered dose immediately following and for a period of time after each dose were recorded.

### Experimental Design

The first dose level of the derivative formulation was administered and mice observed for any effects for approximately 10 min before proceeding to the next higher dose. As doses were tolerated, they were increased. For example, 20 mg/kg, 40 mg/kg and 80 mg/kg was an exemplary progression, depending on observations after each dose.

The following study design was performed for the selected bacterial strains (A. baumannii, E. coli and P. aeruginosa) shown below. Dose selections of derivatives were determined pending the MTD studies. There were 3 dose groups for each test derivative.

TABLE 2

| | | Treatments | | | | Explant | | |
|---|---|---|---|---|---|---|---|---|
| | Test | Dose | | | Regimen | (post-dose) | | |
| Group | derivative | (mg/kg) | Volume | Route | (post-infection) | (hr) | | # |
| 1 | TA1 | | 10 mL/kg | IP | +1 hr | +24 | hr | 3 |
| 2 | | | | | | | | 3 |
| 3 | TA2 | | 10 mL/kg | IP | +1 hr | +24 | hr | 3 |
| 4 | | | | | | | | 3 |
| 5 | TA3 | | 10 mL/kg | IP | +1 hr | +24 | hr | 3 |
| 6 | | | | | | | | 3 |
| 7 | TA4 | | 10 mL/kg | IP | +1 hr | +24 | hr | 3 |
| 8 | | | | | | | | 3 |
| 9 | Positive | xxx | | | | | | 3 |
| | Control* | | | | | | | |
| 10 | Infection | na | na | na | na | +24 | hr | 3 |
| 11 | Controls | | | | | +1 | hr | 3 |

*Positive Controls depend on the bacterial strain and are:
P. aeruginosa UNT202-1: Levofloxacin 200 mg/kg
E. coli UNT156-1: Cefepime 64 mg/kg
A. baumamii UNT190-1: Tigecycline 50 mg/kg

### Sampling and Data Analysis

Mice were euthanized by CO₂ inhalation and thigh samples taken in accordance with the indicated times in the table above. Thighs were aseptically removed, placed in 1 ml-2 ml sterile PBS, homogenized, 10-fold serially diluted in PBS and plated on LB agar to determine CFU counts. Plates were incubated 18 hr-24 hr at 37° C. prior to counting.

The number of colonies observed was converted to CFU/thigh by multiplying the number of colonies by the volume of the thigh homogenate spoiled and the dilution at which the colonies were counted (5-50 colonies/spot). All count data were transformed into log₁₀ CFU/thigh for calculation of means and standard deviations

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims

We claim:

1. A formulation comprising:
a nucleoside analog inhibitor of inosine monophosphate dehydrogenase (IMPDH); and
a pharmaceutically acceptable carrier,
wherein the derivative is not:

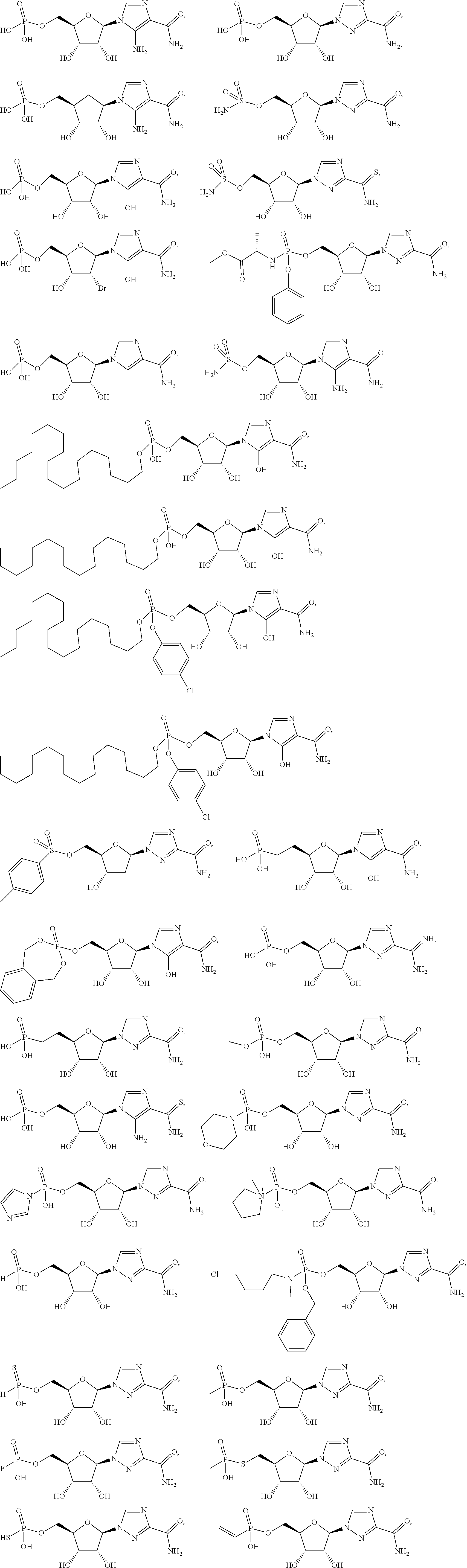

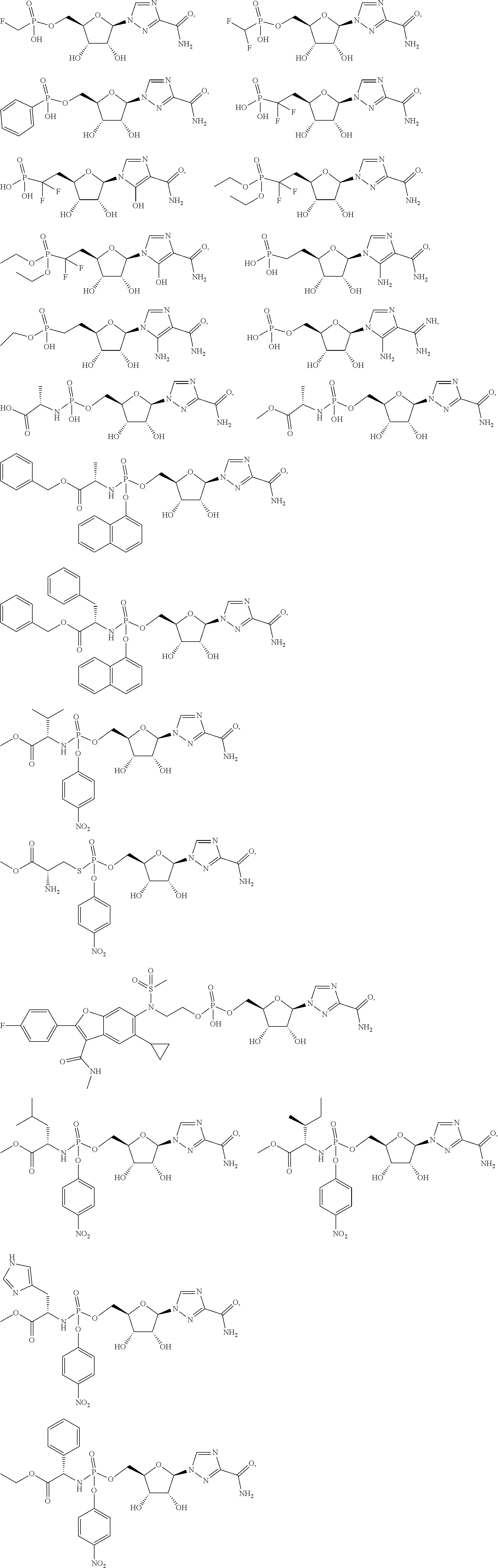

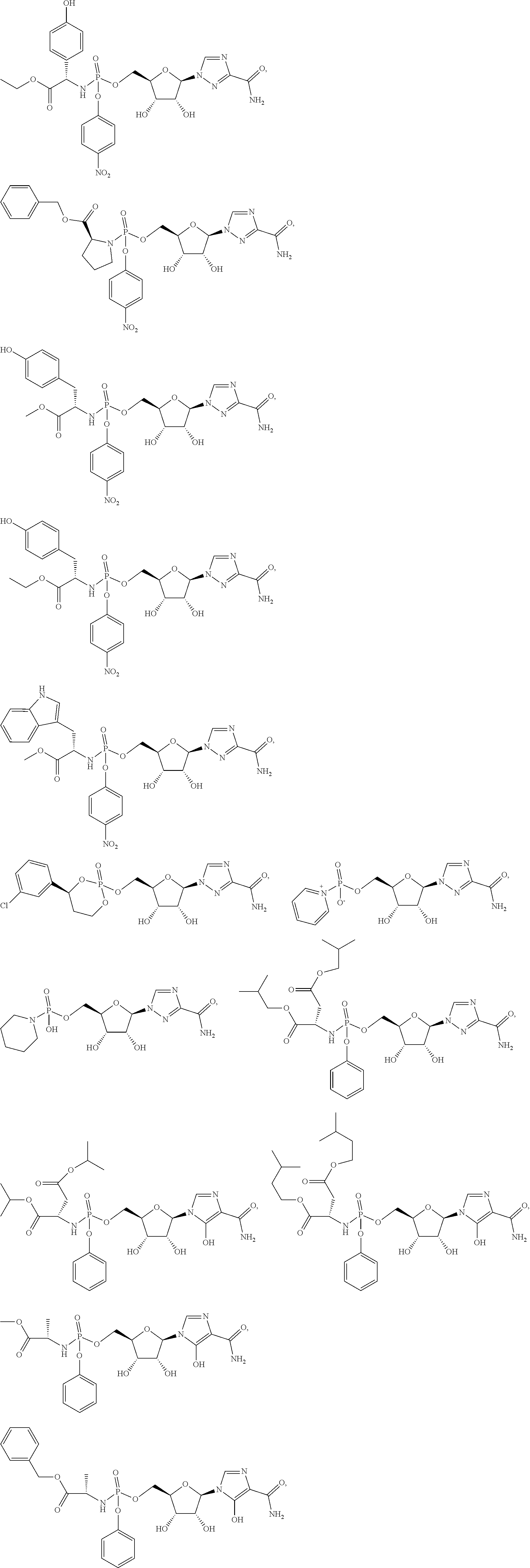

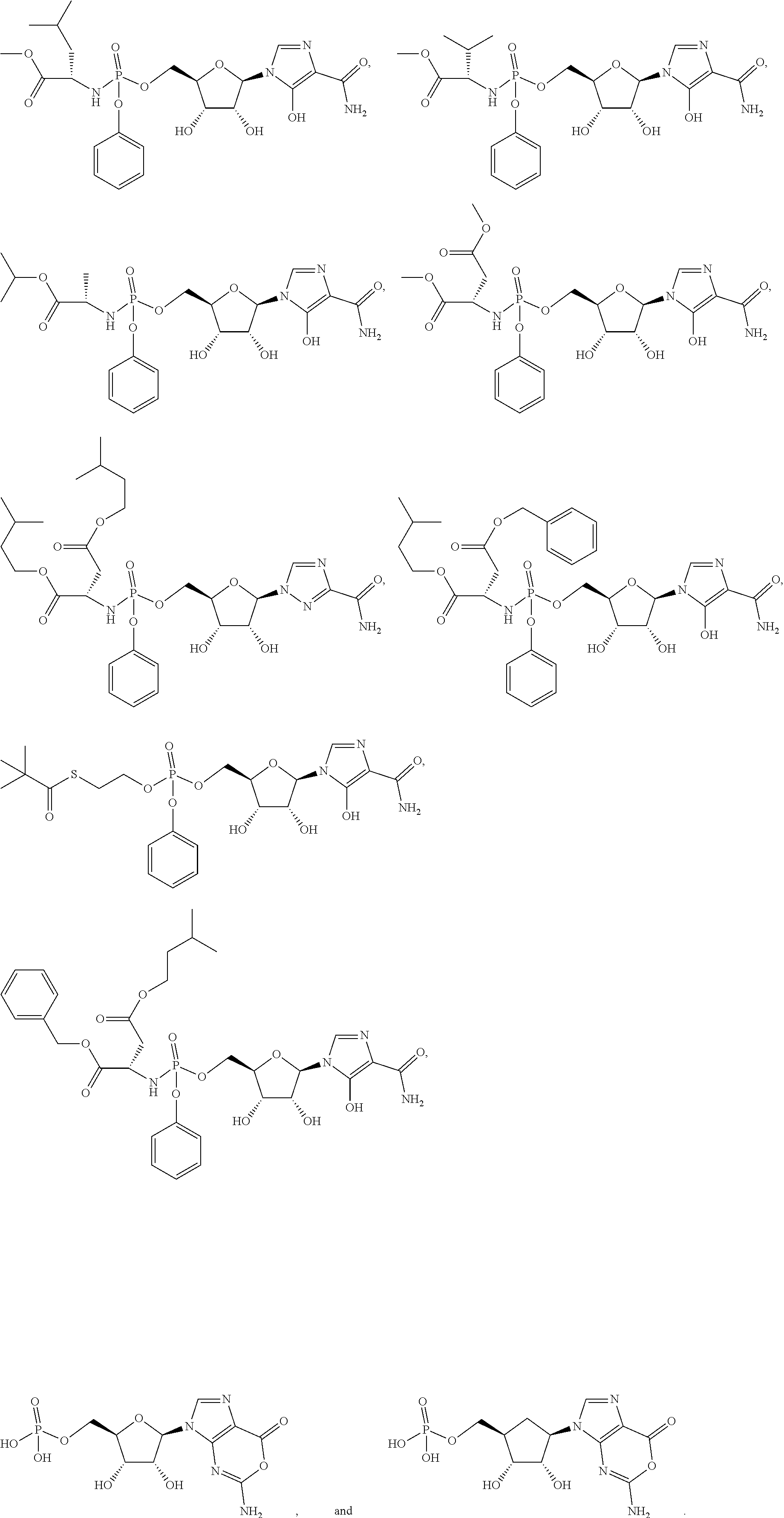

2. The formulation of claim 1, wherein the nucleoside analog inhibitor is a compound of Formula 1 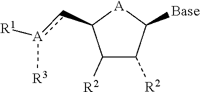
or a pharmaceutically acceptable salt thereof, wherein:
Base is selected from the group consisting of 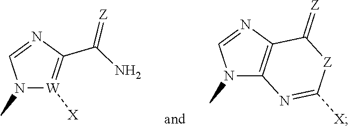
A is selected from the group consisting of —CH—, —CH₂—, —N—, —NH—, —O—, —SO₂R³—, and —S—;
W is selected from the group consisting of —C—, —CH—, —CH₂—, —N—, and —NH₂—;
X is, independently at each occurrence, selected from the group consisting —OH, —SH, —NH₂ halogen;
Y is selected from the group consisting of —OH, —SH, —NH₂, and —N₃;
Z is selected from the group consisting of O, S, and;
R¹ is selected from the group consisting of —PA₁O₂(R³)₂, — and —SO₂R³;
R² is, independently at each occurrence, selected from the group consisting of —OH, —NH₂, and —N₃;
R³ is, independently at each occurrence, selected from the group consisting of —H, —C₁₋₆ alkyl, —C₁₋₆ alkenyl, —C₁₋₆ allyl —C₆₋₁₀ aryl, and —N(R⁴)₂, wherein —C₁₋₆ alkyl is optionally substituted with one or more halo;
R⁴ is, independently at each occurrence, selected from the group consisting of —H, —C₁₋₆ alkyl, —C₁₋₆ alkenyl, —C₁₋₆ allyl and —C₆₋₁₀ aryl; and [symbol]
is an optional bond, and

3. The formulation of claim 2, wherein the nucleoside analog inhibitor is a compound of Formula II: 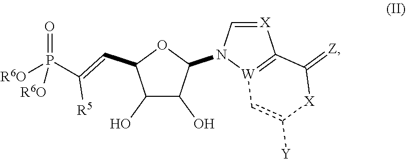
a compound of Formula III: 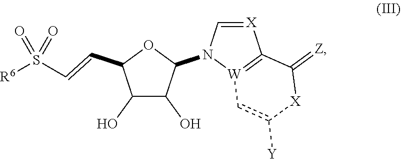
a compound of Formula IV: 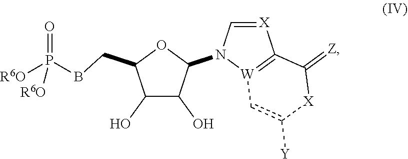
a compound of Formula V: 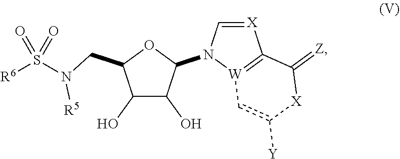
or a pharmaceutically acceptable salt of a compound of Formula II, Formula III, Formula IV, or Formula V,
wherein:
W is selected from the group consisting of —C—, —CH₂—, —N—, and —NH₂—;
X is, independently at each occurrence, selected from the group consisting of —O—, —OH, —S—, —SH, —NH—, —NH₂, —CH₂—, and —CH₃;
Y is selected from the group consisting of —OH, —SH, —NH₂, and —N₃;
Z is selected from the group consisting of O, S, and NH;
B is selected from the group consisting of S, O, NH, and NR⁵;
R⁵ is selected from the group consisting of —H, halo, —C₁₋₆ alkyl, —C₁₋₆ alkenyl, and —C₆₋₁₀ aryl;
R⁶ is, independently at each occurrence, selected from the group consisting of —H, —C₁₋₆ alkyl, —C₁₋₆ alkenyl, —C₆₋₁₀ aryl, —CH₂—C₆₋₁₀ aryl, —O—C₆₋₁₀ aryl, —N(C₁₋₆ alkyl)₂, —NH(C₁₋₆ alkyl), and —NH₂, wherein —C₆₋₁₀ aryl is optionally substituted with one or more R⁷;
or alternatively, each R⁶, together with the atom to which they are attached, can form C₃₋₁₂ heterocycle or C₃₋₁₂ heteroaryl, wherein heterocycle or heteroaryl is optionally substituted with one or more R⁷;
R⁷ is selected from the group consisting of halo, —C₁₋₆ alkyl, —C₁₋₆ alkenyl, —C₆₋₁₀ aryl, —OC₁₋₆ alkyl, —N(C₁₋₆ alkyl)₂, —NH(C₁₋₆ alkyl), —NH₂, and —OH; and [symbol]
is an optional bond.

4. The formulation of claim 3, wherein the compound of Formula I, II, III, IV, or V is a compound have a structure selected from the group consisting of:

| Structure | |
|---|---|
| No. | Structure |
| 1 | 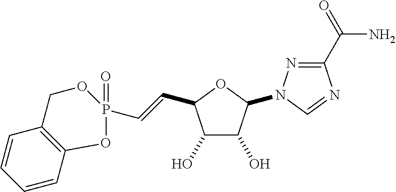 [SMILES: NC(=O)c1ncn([C@@H]2O[C@H](/C=C/P3(=O)OCc4ccccc4O3)[C@@H](O)[C@H]2O)n1] |
| 2 | 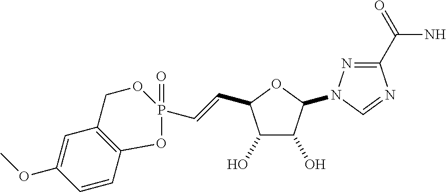 [SMILES: COc1ccc2c(c1)COP(=O)(/C=C/[C@H]1O[C@@H](n3cnc(C(N)=O)n3)[C@H](O)[C@@H]1O)O2] |
| 3 | 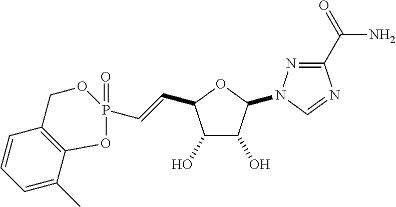 [SMILES: Cc1cccc2c1OP(=O)(/C=C/[C@H]1O[C@@H](n3cnc(C(N)=O)n3)[C@H](O)[C@@H]1O)OC2] |
| 4 | 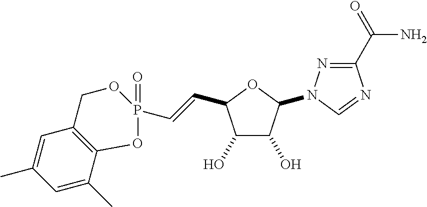 [SMILES: Cc1cc(C)c2c(c1)COP(=O)(/C=C/[C@H]1O[C@@H](n3cnc(C(N)=O)n3)[C@H](O)[C@@H]1O)O2] |
| 5 | 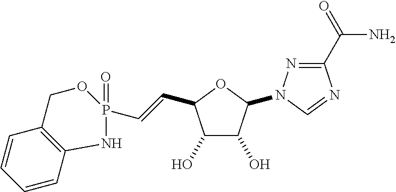 [SMILES: NC(=O)c1ncn([C@@H]2O[C@H](/C=C/P3(=O)Nc4ccccc4CO3)[C@@H](O)[C@H]2O)n1] |
| 6 | 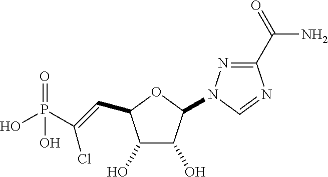 |
| 7 | 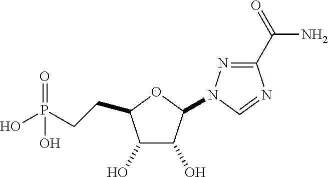 |
| 8 | 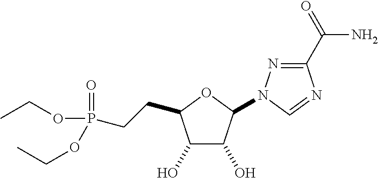 |
| 9 | 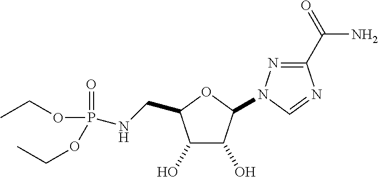 |
| 10 | 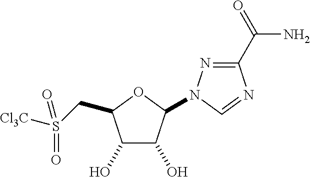 |
| 11 | 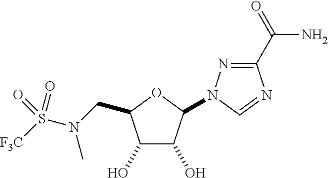 |
| 12 | 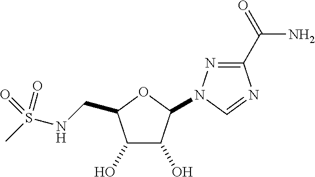 |
| 13 | 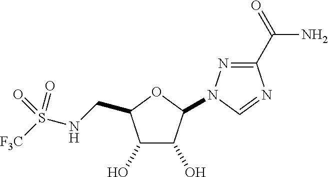 |
| 14 | 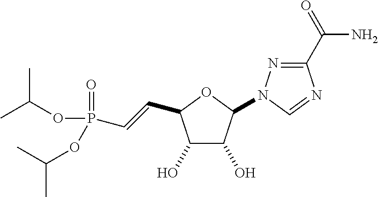 |
| 15 | 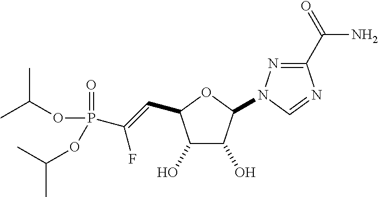 |
| 16 | 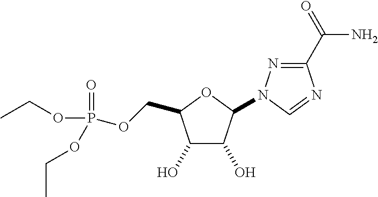 |
| 17 | 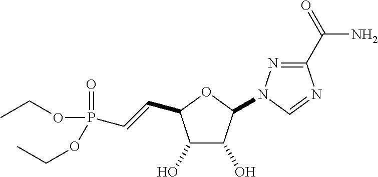 |
| 18 | 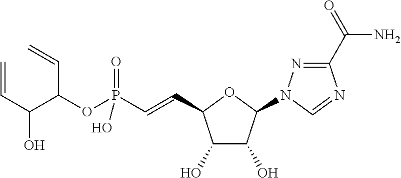 [SMILES: C=CC(O)C(C=C)OP(=O)(O)/C=C/[C@H]1O[C@@H](n2cnc(C(N)=O)n2)[C@H](O)[C@@H]1O] |
| 19 | 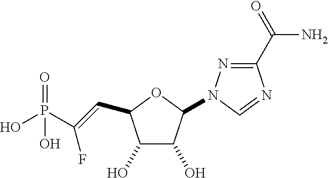 |
| 20 | 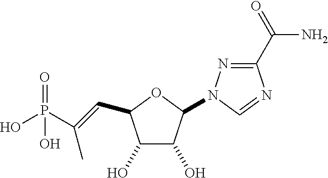 |
| 21 | 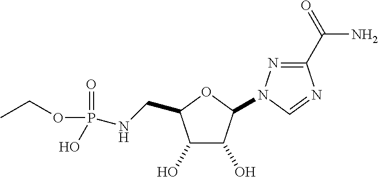 |
| 22 | 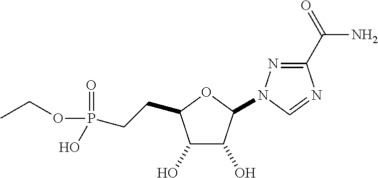 |
| 23 | 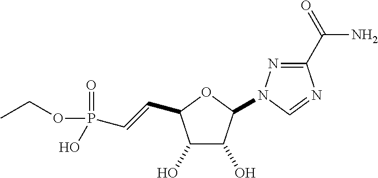 |
| 24 | 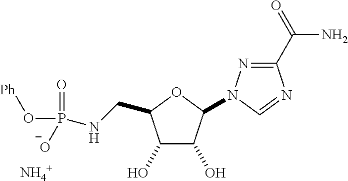 |
| 25 | 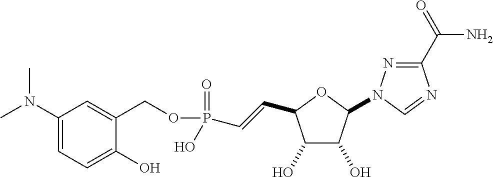 |
| 26 | 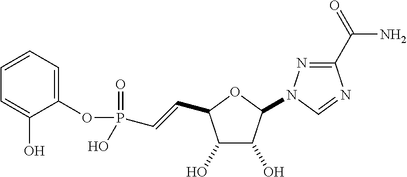 |
| 27 | 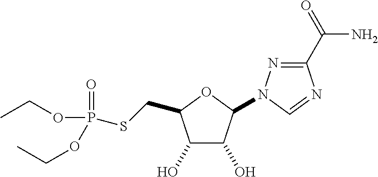 |
| 28 | 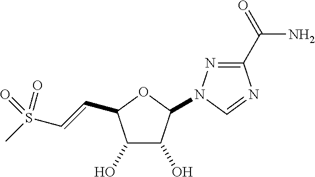 |
| 29 | 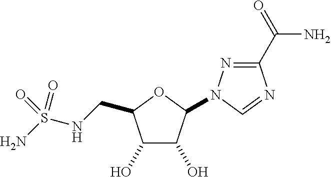 |
| 30 | 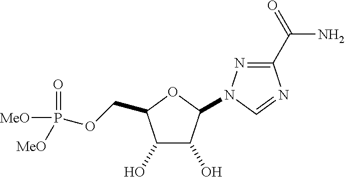 |
| 31 | 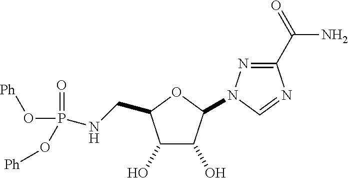 |
| 32 | 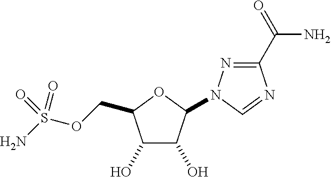 |
| 33 | 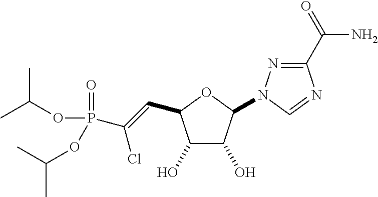 |
| 34 | 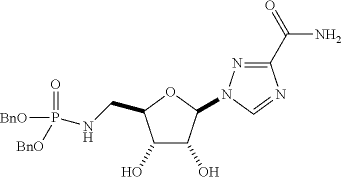 |
| 35 | 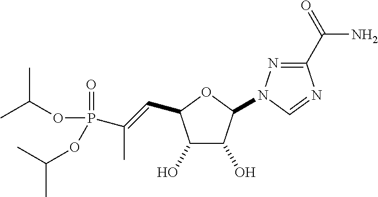 |
| 36 | 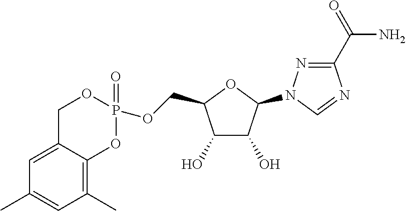 [SMILES: Cc1cc(C)c2c(c1)COP(=O)(OC[C@H]1O[C@@H](n3cnc(C(N)=O)n3)[C@H](O)[C@@H]1O)O2] |

or a pharmaceutically acceptable salt thereof.

5. A method of treating an infection in a mammal, comprising administering to the mammal a therapeutically effective amount of an anti-infective formulation such that the infection is reduced,
the anti-infective formulation comprising a nucleoside analog inhibitor of inosine monophosphate dehydrogenase (IMPDH) and a pharmaceutically acceptable carrier, wherein the derivative is not

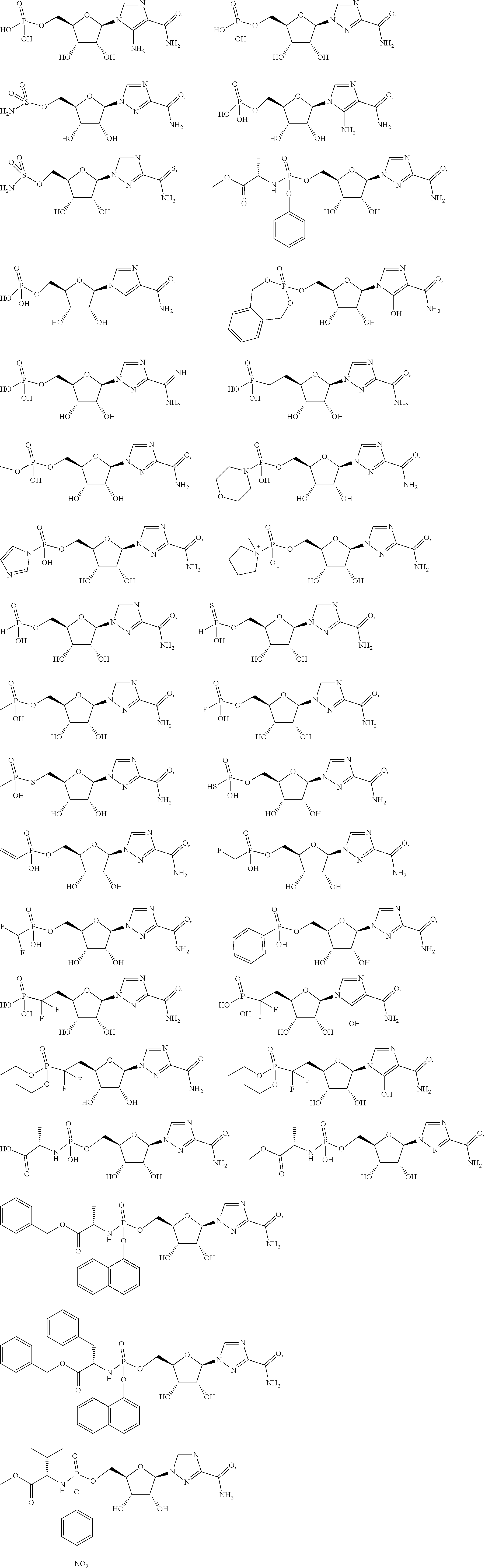

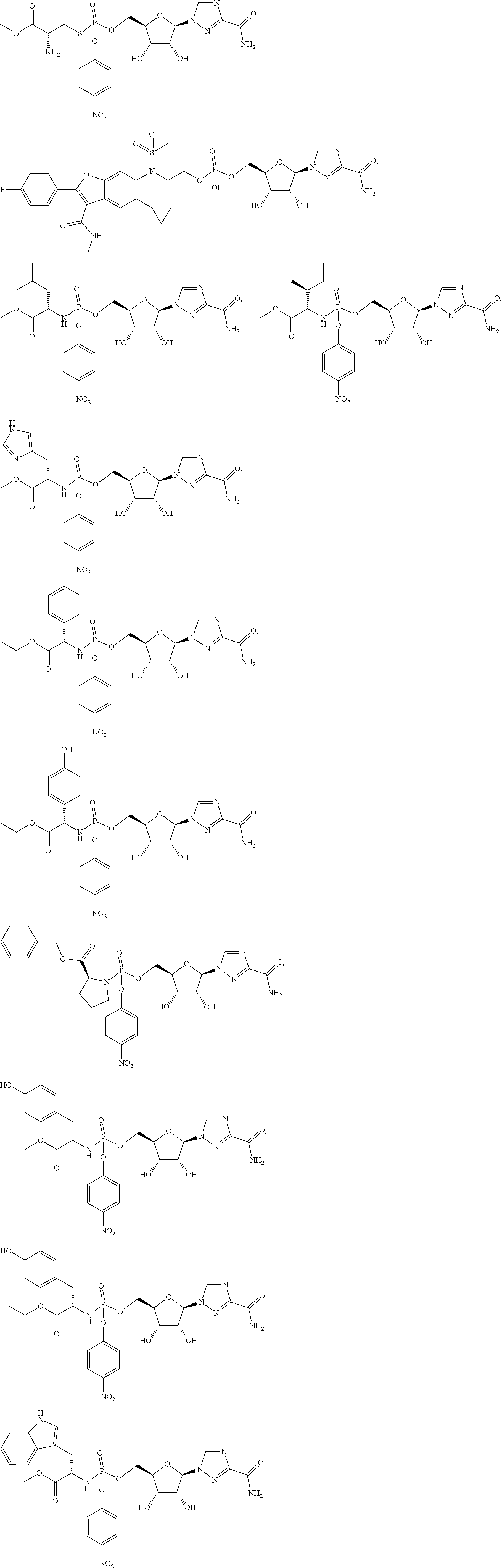

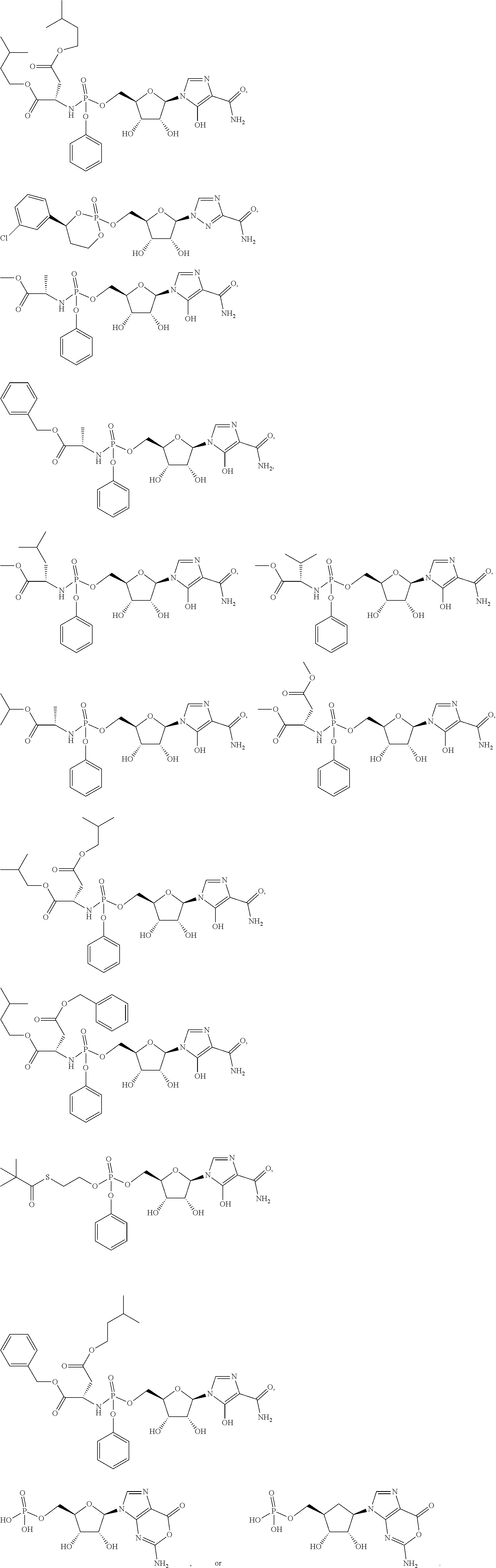

6. The method of claim 5, wherein the infection is a bacterial infection, a fungal infection, a viral infection, a yeast infection, a multicellular parasitic infection, or a protozoan infection.

7. The method of claim, 5, wherein the infection is a bacterial infection.

8. The method of claim 7, wherein the infection is a Gram positive or Gram negative bacterial bacteria.

9. The method of claim 7, wherein the bacterial infection is an infection by Mycobacterium tuberculosis, Mycobacterium avium, Mycobacterium intracellulare, or Mycobacterium leprae.

10. A method of inhibiting the growth and/or proliferation of an infective organism, comprising contacting the organism with a growth and/or proliferation-inhibiting amount of an inti-infective formulation,
the anti-infective formulation comprising a nucleoside analog inhibitor of inosine monophosphate dehydrogenase (IMPDH) and a pharmaceutically acceptable carrier,
wherein the derivative is not 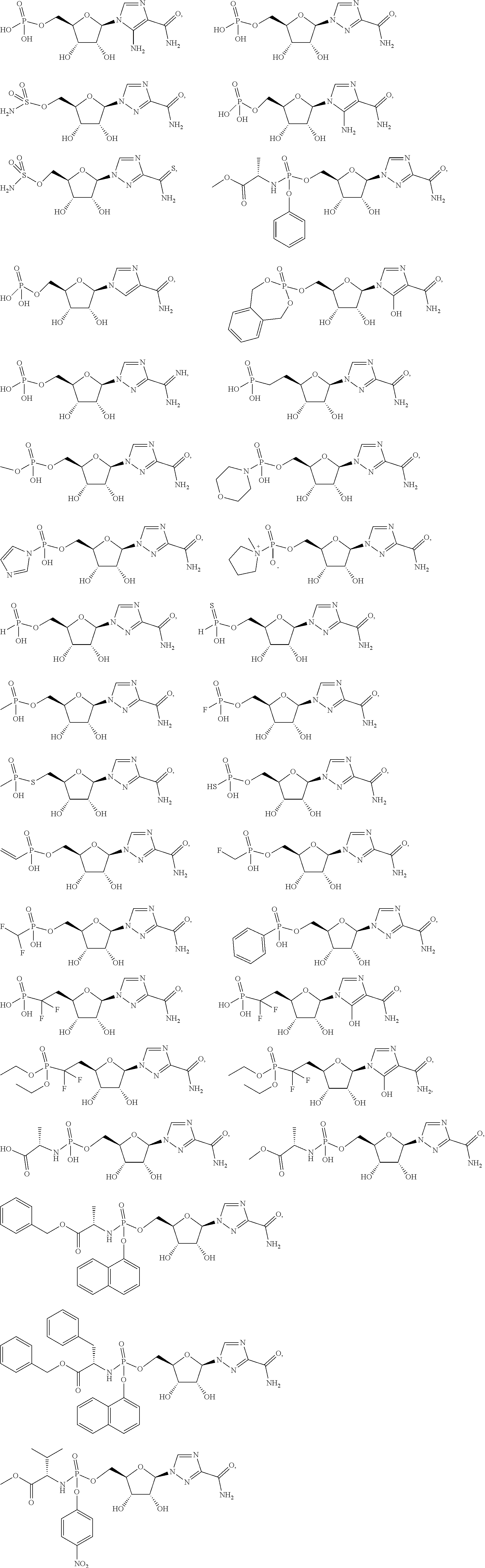 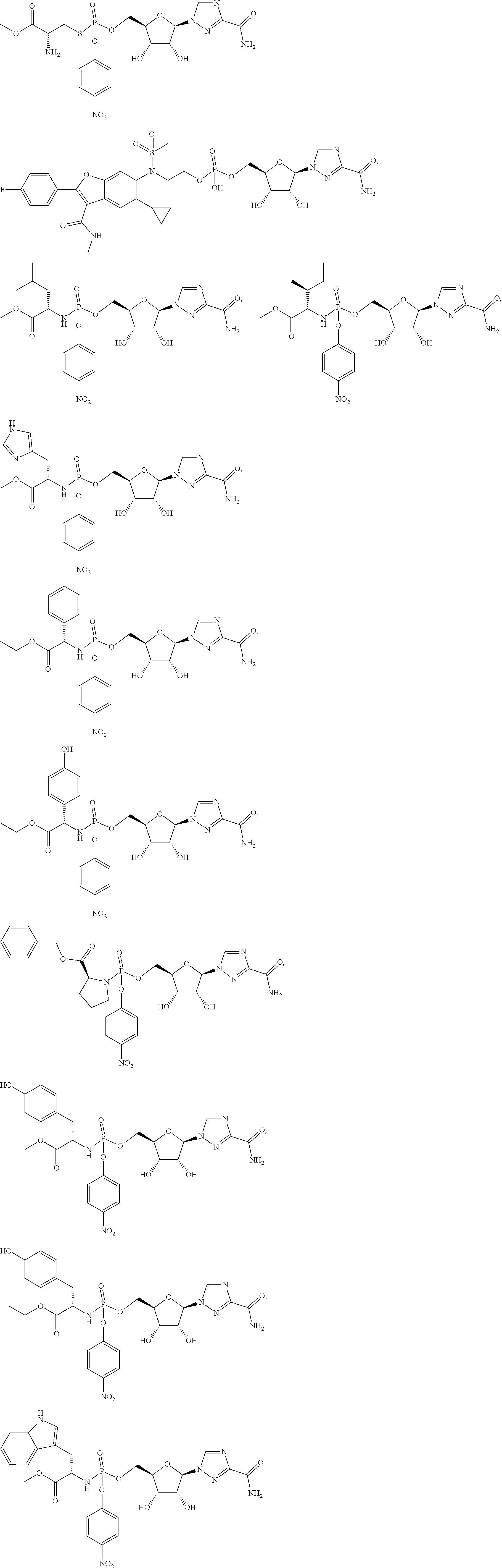 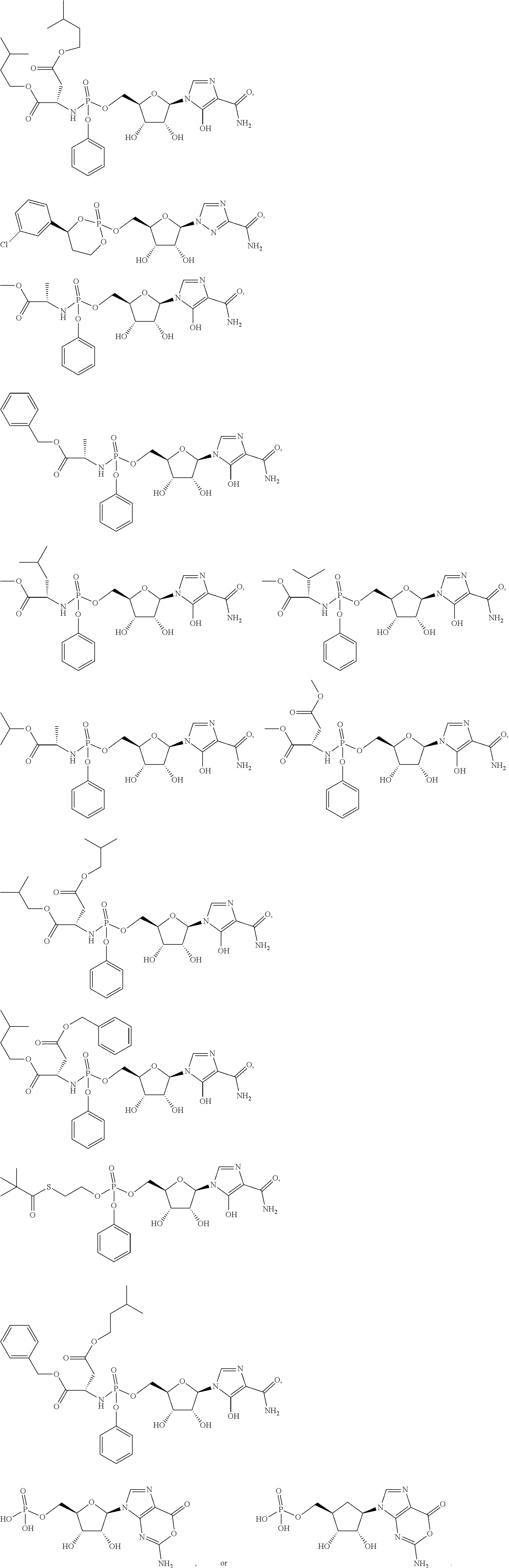
and wherein the infective organism is a bacterium, a fungus, a yeast, a multicellular parasite, or a protozoan.
wherein the nucleoside analog inhibitor is not a known anti-infective compound, and
wherein the infective organism is a bacterium, a fungus, a yeast, a multicellular parasite, or a protozoan.
